(19) United States
(12) Patent Application Publication
(10) Pub. No.: US 2023/0159426 A1
(43) Pub. Date: May 25, 2023

(54) LILY OF THE VALLEY ODORANT

(71) Applicant: Firmenich SA, Satigny (CH)

(72) Inventors: George LEM, Satigny (CH); Vincent HARRACA, Satigny (CH)

(21) Appl. No.: 17/908,525

(22) PCT Filed: Apr. 12, 2021
(86) PCT No.: PCT/EP2021/059459

§ 371 Date: Aug. 31, 2022

(30) Foreign Application Priority Data
Apr. 14, 2020 (EP) 20169294.4

(51) Int. Cl. C07C 47/225; A61K 8/33; A61Q 13/00; A61Q 5/02; A61Q 19/10; A61Q 15/00; A61Q 17/02; A01P 1/00; A01P 17/00; A01N 35/02; A61K 8/04; C11B 9/00; C11D 3/50

(52) U.S. Cl. CPC C07C 47/225; A61K 8/33; A61Q 13/00; A61Q 5/02; A61Q 19/10; A61Q 15/00; A61Q 17/02; A01P 1/00; A01P 17/00; A01N 35/02; A61K 8/046; C11B 9/003; C11D 3/50; C07C 2601/10; A61K 2800/262; A61K 2800/436; A61K 2800/77; A61K 2800/874

(57) ABSTRACT

The present invention relates to the field of perfumery. More particularly, it relates to a compound of formula (I) as defined herein below, and its uses as perfuming ingredient, to control arthropods or as antimicrobial ingredient. Therefore, following what is mentioned herein, the present invention comprises the invention compound as part of a perfuming composition or of a perfumed consumer product. Moreover, the present invention relates to a properfume compound suitable to release the compound of formula (I).

### TECHNICAL FIELD

The present invention relates to the field of perfumery. More particularly, it relates to a compound of formula (I) as defined herein below, and its uses as perfuming ingredient, to control arthropods or as antimicrobial ingredient. Therefore, following what is mentioned herein, the present invention comprises the invention compound as part of a perfuming composition or of a perfumed consumer product. Moreover, the present invention relates to a properfume compound suitable to release the compound of formula (I).

### BACKGROUND OF THE INVENTION

Some of the most sought-after ingredients in the perfumery field are the ones imparting a floral impression and especially a lily of the valley odor. Adding to its value is the observation that this delicate floral odor does not survive even the mildest of extraction methods to yield an essential oil. Said note is very appreciated and used in a multitude of perfumed consumer products. For many decades, a lot of effort has been invested in finding compounds possessing this very complex white floral odor, especially since the use of Lilial® (2-methyl-3-[4-(2-methyl-2-propanyl)phenyl]propanal, trademark from Givaudan-Roure SA, Vernier, Suisse) representing one of the most valuable perfuming ingredients with a lily of the valley and watery connotation, has been limited due to various reasons.

There is a need to develop novel perfuming ingredients conferring a floral odor note being as close as possible to the natural odor of the lily of the valley blossom.

WO2018114844 reports 3-((S)-4-isopropylcyclohex-1-en-1-yl)-2-methylpropanal, a compound imparting odor notes of the lily of the valley type with a white peach connotation.

The present invention provides a novel perfumery ingredient imparting lily of the valley note, by using compounds of formula (I). The prior art document mentioned above does not disclose the compounds of formula (I) or the organoleptic properties of the compounds of formula (I).

### SUMMARY OF THE INVENTION

The invention relates to a compound of formula (I) imparting an odor of floral type, in particular lily of the valley (also named muguet) which is much appreciated in perfumery.

So, a first object of the present invention is a compound of formula

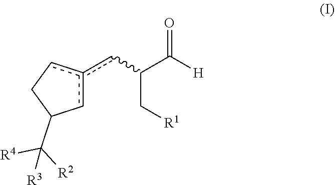

in the form of any one of its stereoisomers or as a mixture thereof, wherein one dotted line represents a carbon-carbon double bond and the other dotted lines represent a carbon-carbon single bond; R¹ represents a hydrogen atom or a C₁₋₂ alkyl group; R² represents a C₁₋₄ alkyl group or a C₂₋₄ alkenyl group; R³ represents a hydrogen atom or a C₁₋₃ alkyl group or a C₂₋₃ alkenyl group; R⁴ represents a hydrogen atom or R⁴ represents a hydrogen atom or a methyl group when R² and R³ represent a methyl group; or R³ and R⁴, when taken together, represent a methylidene group; or R² and R³, when taken together, represent a C₄₋₉ alkanediyl or a C₄₋₉ alkenediyl group; R², R³ and R⁴, when taken together, represent a phenyl group optionally substituted by a C₁₋₃ alkyl group.

A second object of the present invention is a use as perfuming ingredient of a compound of formula (I) as defined above.

A third object of the present invention is a method to confer, enhance, improve or modify the odor properties of a perfuming composition or of a perfumed article, which method comprises adding to said composition or article an effective amount of at least one compound of formula (I) as defined above.

Another object of the present invention is a properfume compound suitable to release the compound of formula

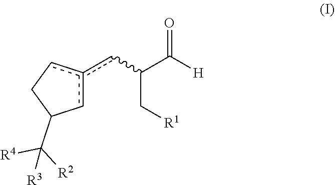

in the form of any one of its stereoisomers or as a mixture thereof, wherein one dotted line represents a carbon-carbon double bond and the other dotted lines represent a carbon-carbon single bond; R¹ represents a hydrogen atom or a C₁₋₂ alkyl group; R² represents a C₁₋₄ alkyl group or a C₂₋₄ alkenyl group; R³ represents a hydrogen atom or a C₁₋₃ alkyl group or a C₂₋₃ alkenyl group; R⁴ represents a hydrogen atom or R⁴ represents a hydrogen atom or a methyl group when R² and R³ represent a methyl group; or R³ and R⁴, when taken together, represent a methylidene group; or R² and R³, when taken together, represent a C₄₋₉ alkanediyl or a C₄₋₉ alkenediyl group; R², R³ and R⁴, when taken together, represent a phenyl group optionally substituted by a C₁₋₃ alkyl group.

Another object of the present invention is a perfuming composition comprising
i) at least one compound of formula (I) and/or at least one properfume compound, as defined above;
ii) at least one ingredient selected from the group consisting of a perfumery carrier and a perfumery base; and
iii) optionally at least one perfumery adjuvant.

Another object of the present invention is a perfumed consumer product comprising at least one compound of formula (I), and/or at least one properfume compound or a composition as defined above.

Another object of the present invention is a method to confer, enhance, improve or modify the arthropod control properties of an arthropod control composition or of an arthropod control article, which method comprises adding to said composition or article an effective amount of at least a compound of formula (I) as defined above.

A further object of the present invention is use of a compound of formula (I) as defined above to control arthropods, preferably insects.

A further object of the present invention provides an antimicrobial composition for use in a perfumed consumer product comprising one or more compounds of formula (I) as defined above.

A further object of the present invention provides the use of composition comprising one or more compounds of formula (I) as an antimicrobial composition.

A further object of the present invention provides a method to reduce, arrest, or inhibit the growth rate of bacterial cells or to inactivate or kill bacterial cells which method comprises adding an effective amount of at least a compound of formula (I) as defined above to a composition or consumer product, or contacting or treating the surface with an effective amount of at least a compound of formula (I) as defined above.

A further object of the present invention provides a non-therapeutic method of reducing, arresting or inhibiting the growth rate of bacterial cells or inactivating or killing bacterial cells, the method comprising treating a substrate comprising microbes with an effective amount of at least a compound of formula (I) as defined above

A further object of the present invention provides a perfuming composition comprising:

a composition comprising compound(s) of formula (I) wherein the composition is present in an amount sufficient to provide an antimicrobial effect;

at least one ingredient selected from the group consisting of a perfumery carrier and a perfumery base; and

optionally at least one perfumery adjuvant.

### DESCRIPTION OF THE INVENTION

Surprisingly, it has now been discovered that a compound of formula (I) imparts a lily of the valley note with green and ozone aspect. In addition, the invention compound possesses an impactful top note very much sought providing a bloom effect.

So, a first object of the present invention is a compound of formula

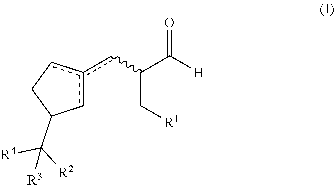

in the form of any one of its stereoisomers or as a mixture thereof, wherein one dotted line represents a carbon-carbon double bond and the other dotted lines represent a carbon-carbon single bond; R¹ represents a hydrogen atom or a C₁₋₂ alkyl group; R² represents a C₁₋₄ alkyl group or a C₂₋₄ alkenyl group; R³ represents a hydrogen atom or a C₁₋₃ alkyl group or a C₂₋₃ alkenyl group; R⁴ represents a hydrogen atom or R⁴ represents a hydrogen atom or a methyl group when R² and R³ represent a methyl group; or R³ and R⁴, when taken together, represent a methylidene group; or R² and R³, when taken together, represent a C₄₋₉ alkanediyl or C₄₋₉ alkenediyl group; R², R³ and R⁴, when taken together, represent a phenyl group optionally substituted by one or two C₁₋₃ alkyl groups.

Said compound can be used as perfuming ingredient, for instance to impart odor notes of the lily of the valley type with green and ozone facets.

According to any one of the above embodiments of the invention, said compounds (I) are C₁₁-C₁₉ compounds.

For the sake of clarity, by the expression “any one of its stereoisomers or a mixture thereof”, or the similar, it is meant the normal meaning understood by a person skilled in the art, i.e. that the compound of formula (I) can be a pure enantiomer (if chiral) or diastereomer (e.g. the double bond is in a conformation E or Z). In other words, the compound of formula (I) may possess several stereocenters and each of said stereocenter can have two different stereochemistries (e.g. R or S). The compound of formula (I) may even be in the form of a pure enantiomer or in the form of a mixture of enantiomers or diastereoisomers. The compound of formula (I) can be in a racemic form or scalemic form. Therefore, the compound of formula (I) can be one stereoisomer or in the form of a composition of matter comprising, or consisting of, various stereoisomers.

The wavy line indicates that the double bond may be in the form of its E or Z isomer or of a mixture thereof when the exocyclic dotted line represents a double bond; e.g. the invention comprises compositions of matter consisting of one or more compounds of formula (I), having the same chemical structure but differing by the configuration of the double bond. In particular, compound (I) can be in the form of a mixture consisting of isomers E and Z and wherein said isomers E represent at least 50% of the total mixture, or even at least 75% (i.e a mixture E/Z comprised between 75/25 and 100/0).

The term “methylidene” designates a CH₂=group; i.e. when R³ and R⁴, taken together, represent a methylidene group, the compound of formula (I) is a compound of formula

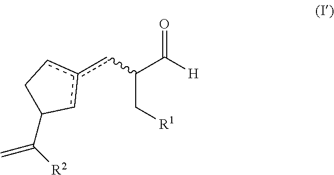

in the form of any one of its stereoisomers or as a mixture thereof, wherein the dotted lines, R¹ and R² have the same meaning as defined above.

For the sake of clarity, by the expression “wherein one dotted line represents a carbon-carbon double bond and the other dotted lines represent a carbon-carbon single bond”, or the similar, it is meant the normal meaning understood by a person skilled in the art, i.e. that the whole bonding (solid and dotted line) between the carbon atoms connected by said dotted line is a carbon-carbon single or double bond. In other words, compound of formula (I) may be

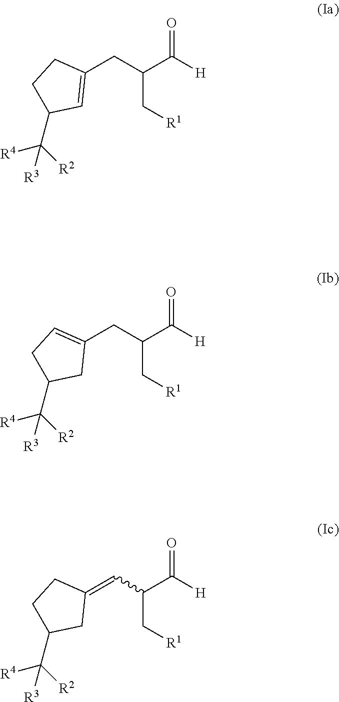

in the form of any one of its stereoisomers or as a mixture thereof, wherein R¹, R², R³ and R⁴ have the same meaning as defined above.

According to any embodiments of the invention, the invention compound may be in a form of a composition of matter comprising compounds of formula (Ia), (Ib) and (Ic).

More particularly, the composition of matter may comprise from 23 to 77% w/w of compound of formula (Ia), from 23 to 77% w/w of compound of formula (Ib) and from from 0.1 to 25% w/w of compound of formula (Ic), the percentage being relative to the total weight of the composition of matter.

For the sake of clarity, by the expression “R² and R³, when taken together, represent a C₄₋₉ alkanediyl or C₄₋₉ alkenediyl group” or similar, it is meant the normal meaning understood by a person skilled in the art, i.e. a divalent group formed from alkane or alkene by removal of two hydrogen atoms. The alkanediyl or alkenediyl group may be linear or branched. In other words, R² and R³, when taken together, form a cycloalkyl or a cycloalkenyl group, each optionally substituted by an alkyl or alkenyl group.

According to any embodiments of the invention, the invention's compound is of formula

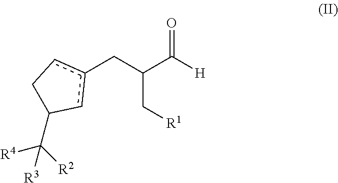

in the form of any one of its stereoisomers or as a mixture thereof, wherein one dotted line represents a carbon-carbon double bond and the other dotted line represents a carbon-carbon single bond; R¹, R², R³ and R⁴ have the same meaning as defined above.

According to any embodiments of the invention, the invention's compound is of formula

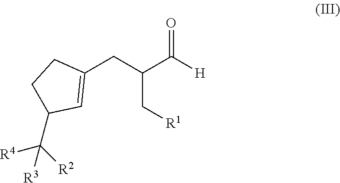

in the form of any one of its stereoisomers or as a mixture thereof, wherein R¹, R², R³ and R⁴ have the same meaning as defined in claim 1.

According to any embodiments of the invention, R¹ may be a hydrogen atom or a methyl group. Particularly, R¹ may be a hydrogen atom.

According to any embodiments of the invention, R⁴ may be a hydrogen atom.

According to any embodiments of the invention, R³ may be a hydrogen atom or a C₁₋₃ alkyl group. Particularly, R³ may be a hydrogen atom, a methyl or an ethyl group. Even more particularly, R³ may be a hydrogen atom or a methyl group.

According to any embodiments of the invention, R² may be a C₁₋₄ alkyl group. Particularly, R² may be a methyl, ethyl or propyl group.

According to a particular embodiment of the invention, R² and R³ are taken together and may represent a C₄₋₆ alkanediyl or C₄₋₆ alkenediyl group. Particularly, R² and R³ are taken together and may form a cyclopentyl, a cyclohexyl, a cyclopentenyl or a cyclohexenyl group.

According to a particular embodiment of the invention, R², R³ and R⁴ are taken together and may represent a phenyl group or a tolyl group.

As specific examples of the invention's compounds, one may cite, as non-limiting example, a composition of matter comprising 3-(3-isopropylcyclopent-1-en-1-yl)-2-methylpropanal and 3-(4-isopropylcyclopent-1-en-1-yl)-2-methylpropanal imparting floral notes and in particular lily of the valley notes. Said invention composition of matter possesses the green, aldehydic, ozone connotation of the lily of the valley. Moreover, said compound imparts a very impactful top note very much sought. Said invention's compound provides a bloom effect to the perfuming composition or to the perfumed consumer product wherein it is added.

As other specific, but non-limiting, examples of the invention's compounds, one may cite the following ones in Table 1:

TABLE 1
Invention’s compounds and their odor properties

| Compound structure and name | Odor notes |
|---|---|
| 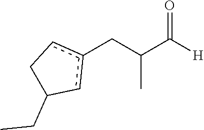 | Aldehydic, green, Lily of the valley |
| 3-(3-ethyl-1-cyclopenten-1-yl)-2- | |
| methylpropanal and | |
| 3-(4-ethyl-1-cyclopenten- | |
| 1-yl)-2-methylpropanal | |
| 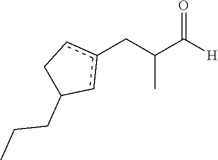 | Aldehydic, green, Lily of the valley |
| 3-(3-propyl-1-cyclopenten-1-yl)-2- | |
| methylpropanal and | |
| 3-(4-propyl-1-cyclopenten- | |
| 1-yl)-2-methylpropanal | |
| 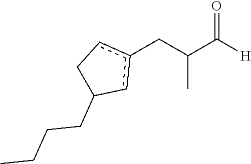 | Aldehydic, ozone, Lily of the valley |
| 3-(3-butyl-1-cyclopenten-1-yl)-2- | |
| methylpropanal and | |
| 3-(4-butyl-1-cyclopenten- | |
| 1-yl)-2-methylpropanal | |
| 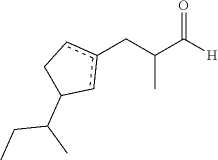 | Aldehydic, ozone, Lily of the valley |
| 3-(3-sec-butyl-1-cyclopenten-1-yl)-2- | |
| methylpropanal and 3-(4-sec-butyl-1- | |
| cyclopenten-1-yl)-2-methylpropanal | |
| 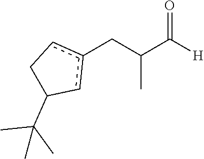 | Aldehydic, ozone, Lily of the valley |
| 3-(3-tert-butyl-1-cyclopenten-1-yl)-2- | |
| methylpropanal and 3-(4-tert-butyl-1- | |
| cyclopenten-1-yl)-2-methylpropanal | |

According to a particular embodiment of the invention, the compounds of formula (I) are 3-(3/4-ethylcyclopent-1-en-1-yl)-2-methylpropanal, 3-(3/4-propylcyclopent-1-en-1-yl)-2-methylpropanal, 3-(3/4-butylcyclopent-1-en-1-yl)-2-methylpropanal, 3-(3/4-isopropylcyclopent-1-en-1-yl)-2-methylpropanal, 3-(3/4-(sec-butyl)cyclopent-1-en-1-yl)-2-methylpropanal and 3-(3/4-(tert-butyl)cyclopent-1-en-1-yl)-2-methylpropanal. Particularly, the compounds of formula (I) is 3-(3/4-isopropylcyclopent-1-en-1-yl)-2-methylpropanal.

When the odor of the invention's compounds is compared with that of the prior art compound 3-((S)-4-isopropylcyclohex-1-en-1-yl)-2-methylpropanal, then the invention's compounds distinguish themselves by clearly stronger green and ozone notes and by lacking the white peach note so characteristic of the prior art compound.

The invention's compounds possess a bloom effect which is not observed for the prior art compound. In other word, the invention compound imparts a more powerful top note that the prior art compound. Said differences lend the invention's compounds and the prior art compounds to be each suitable for different uses, i.e. to impart different organoleptic impressions.

As mentioned above, the invention concerns the use of a compound of formula (I) as a perfuming ingredient. In other words, it concerns a method or a process to confer, enhance, improve or modify the odor properties of a perfuming composition or of a perfumed article or of a surface, which method comprises adding to said composition or article an effective amount of at least a compound of formula (I), e.g. to impart its typical note. Understood that the final hedonic effect may depend on the precise dosage and on the organoleptic properties of the invention's compound, but anyway the addition of the invention's compound will impart to the final product its typical touch in the form of a note, touch or aspect depending on the dosage.

By “use of a compound of formula (I)” it has to be understood here also the use of any composition containing a compound (I) and which can be advantageously employed in the perfumery industry.

Said compositions, which in fact can be advantageously employed as perfuming ingredients, are also an object of the present invention.

Therefore, another object of the present invention is a perfuming composition comprising:
i) as a perfuming ingredient, at least one invention's compound as defined above and/or at least one properfume compound releasing a compound of formula (I);
ii) at least one ingredient selected from the group consisting of a perfumery carrier and a perfumery base; and
iii) optionally at least one perfumery adjuvant.

By “perfumery carrier” it is meant here a material which is practically neutral from a perfumery point of view, i.e. that does not significantly alter the organoleptic properties of perfuming ingredients. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples, solvents such as butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethano, tri-ethyl citrate or mixtures thereof, which are the most commonly used. For the compositions which comprise both a perfumery carrier and a perfumery base, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castor oils such as those known under the trademark Cremophor® RH 40 (origin: BASF).

Solid carrier is meant to designate a material to which the perfuming composition or some element of the perfuming composition can be chemically or physically bound. In general such solid carriers are employed either to stabilize the composition, or to control the rate of evaporation of the compositions or of some ingredients. Solid carriers are of current use in the art and a person skilled in the art knows how to reach the desired effect. However by way of non-limiting examples of solid carriers, one may cite absorbing gums or polymers or inorganic materials, such as porous polymers, cyclodextrins, wood based materials, organic or inorganic gels, clays, gypsum talc or zeolites.

As other non-limiting examples of solid carriers, one may cite encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs- and Geliermittel in Lebensmitteln, Band 2 der Schriftenreihe Lebensmittelchemie, Lebensmittelqualitat, Behr's Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation techniques.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation processes induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof. Alternatively, one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Other resins are the ones produced by the polycondensation of an a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate are preferred.

Some of the seminal literature related to the encapsulation of perfumes by polycondensation of amino resins, namely melamine-based resins with aldehydes includes articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. U.S. Pat. No. 4,396,670, to the Wiggins Teape Group Limited is a pertinent early example of the latter. Since then, many other authors have enriched the literature in this field and it would be impossible to cover all published developments here, but the general knowledge in encapsulation technology is very significant. More recent publications of pertinence, which disclose suitable uses of such microcapsules, are represented for example by the article of K. Bruyninckx and M. Dusselier, ACS Sustainable Chemistry & Engineering, 2019, vol. 7, pages 8041-8054.

By “perfumery base” what is meant here is a composition comprising at least one perfuming co-ingredient.

Said perfuming co-ingredient is not of formula (I). Moreover, by “perfuming co-ingredient” it is meant here a compound, which is used in a perfuming preparation or a composition to impart a hedonic effect. In other words such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients present in the base do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin.

In particular one may cite perfuming co-ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0˜2,7˜]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-methanol, propyl (S)-2-(1,1-dimethylpropoxy)propanoate, 2-methoxynaphthalene, 2,2,2-trichloro phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0^{2,7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl cyclopenten-1-yl)-2-buten-1-ol, methyl cedryl ketone, 5-(2,2,3-trimethyl cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl)butanal.

A perfumery base according to the invention may not be limited to the above mentioned perfuming co-ingredients, and many other of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said co-ingredients may also be compounds known to release in a controlled manner various types of perfuming compounds also known as properfume or profragrance. Non-limiting examples of suitable properfume may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene or a mixture thereof.

By “perfumery adjuvant”, it is meant here an ingredient capable of imparting additional added benefit such as a color, a particular light resistance, chemical stability, etc. A detailed description of the nature and type of adjuvant commonly used in perfuming composition cannot be exhaustive, but it has to be mentioned that said ingredients are well known to a person skilled in the art. One may cite as specific non-limiting examples the following: viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellants, ointments, vitamins and mixtures thereof.

It is understood that a person skilled in the art is perfectly able to design optimal formulations for the desired effect by admixing the above-mentioned components of a perfuming composition, simply by applying the standard knowledge of the art as well as by trial and error methodologies.

An invention's composition consisting of at least one compound of formula (I) and/or at least one properfume releasing the compound of formula (I) and at least one perfumery carrier consists of a particular embodiment of the invention as well as a perfuming composition comprising at least one compound of formula (I) and/or at least one properfume releasing the compound of formula (I), at least one perfumery carrier, at least one perfumery base, and optionally at least one perfumery adjuvant.

According to a particular embodiment, the compositions mentioned above, comprise more than one compound of formula (I) and enable the perfumer to prepare accords or perfumes possessing the odor tonality of various compounds of the invention, creating thus new building block for creation purposes.

For the sake of clarity, it is also understood that any mixture resulting directly from a chemical synthesis, e.g. a reaction medium without an adequate purification, in which the compound of the invention would be involved as a starting, intermediate or end-product could not be considered as a perfuming composition according to the invention as far as said mixture does not provide the inventive compound in a suitable form for perfumery. Thus, unpurified reaction mixtures are generally excluded from the present invention unless otherwise specified.

The invention's compound can also be advantageously used in all the fields of modern perfumery, i.e. fine or functional perfumery, to positively impart or modify the odor of a consumer product into which said compound (I) is added. Consequently, another object of the present invention consists of a perfumed consumer product comprising, as a perfuming ingredient, at least one compound of formula (I) and/or at least one properfume compound as defined below or a perfuming composition as defined above.

The invention's compound can be added as such or as part of an invention's perfuming composition.

For the sake of clarity, “perfumed consumer product” is meant to designate a consumer product which delivers at least a pleasant perfuming effect to the surface or space to which it is applied (e.g. skin, hair, textile, or home surface). In other words, a perfumed consumer product according to the invention is a perfumed consumer product which comprises a functional formulation, as well as optionally additional benefit agents, corresponding to the desired consumer product, and an olfactive effective amount of at least one invention's compound. For the sake of clarity, said perfumed consumer product is a non-edible product.

The nature and type of the constituents of the perfumed consumer product do not warrant a more detailed description here, which in any case would not be exhaustive, the skilled person being able to select them on the basis of his general knowledge and according to the nature and the desired effect of said product.

Non-limiting examples of suitable perfumed consumer products include a perfume, such as a fine perfume, a splash or eau de parfum, a cologne or a shave or after-shave lotion; a fabric care product, such as a liquid or solid detergent, a fabric softener, a liquid or solid scent booster, a fabric refresher, an ironing water, a paper, a bleach, a carpet cleaner, a curtain-care product; a body-care product, such as a hair care product (e.g. a shampoo, a coloring preparation or a hair spray, a color-care product, a hair shaping product, a dental care product), a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or cars) and/or in a public space (halls, hotels, malls, etc.); or a home care product, such as a mold remover, a furnisher care product, a wipe, a dish detergent or a hard-surface (e.g. a floor, bath, sanitary or a window-cleaning) detergent; a leather care product; a car care product, such as a polish, a wax or a plastic cleaner, a pest control article, an insect repellent product.

The proportions in which the compounds according to the invention can be incorporated into the various aforementioned products or compositions vary within a wide range of values. These values are dependent on the nature of the article to be perfumed and on the desired organoleptic effect as well as on the nature of the co-ingredients in a given base when the compounds according to the invention are mixed with perfuming co-ingredients, solvents or additives commonly used in the art.

For example, in the case of perfuming compositions, typical concentrations are in the order of 0.01% to 10% by weight, or even more, of the compounds of the invention based on the weight of the composition into which they are incorporated. In the case of perfumed consumer product, typical concentrations are in the order of 0.0001% to 5% by weight, or even more, of the compounds of the invention based on the weight of the consumer product into which they are incorporated.

Some of the above-mentioned perfumed consumer products may represent an aggressive medium for the invention's compounds, so that it may be necessary to protect the latter from premature decomposition, for example by encapsulation or by chemically binding it to another chemical which is suitable to release the invention's ingredient upon a suitable external stimulus, such as an enzyme, light, heat or a change of pH. A precursor compound able to release a perfuming ingredient under a suitable trigger is also known as properfume or profragrance.

So, another object of the present invention is a properfume compound suitable to release the compound of formula

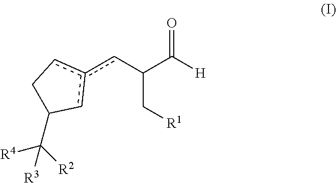

in the form of any one of its stereoisomers or as a mixture thereof, wherein one dotted line represents a carbon-carbon double bond and the other dotted lines represent a carbon-carbon single bond; R¹ represents a hydrogen atom or a C₁₋₂ alkyl group; R² represents a C₁₋₄ alkyl group or a C₂₋₄ alkenyl group; R³ represents a hydrogen atom or a C₁₋₃ alkyl group or a C₂₋₃ alkenyl group; R⁴ represents a hydrogen atom or R⁴ represents a hydrogen atom or a methyl group when R² and R³ represent a methyl group; or R³ and R⁴, when taken together, represent a methylidene group; or R² and R³, when taken together, represent a C₄₋₉ alkanediyl group, a C₄₋₉ alkenediyl; R², R³ and R⁴, when taken together, represent a phenyl group optionally substituted by a C₁₋₃ alkyl group.

According a particular embodiment, the properfume compound may be in the form of an alpha-ketoester (2-oxoacetate). Such a properfume may be obtained by esterification of an alpha-ketoacid or by trans-esterification of an alpha-ketoester and the alcohol of formula

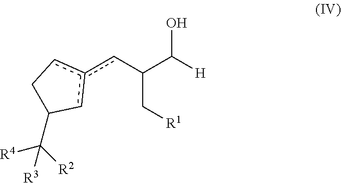

in the form of any one of its stereoisomers or as a mixture thereof, wherein the dotted lines, R¹, R², R³ and R⁴ have the same meaning as defined above.

According to a particular embodiment, the properfume compound may be in the form of an alpha-ketoester of formula

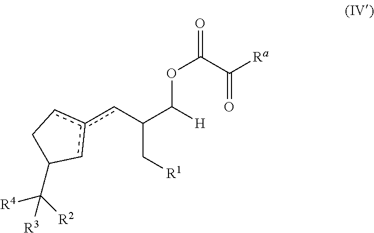

wherein the dotted lines, R¹, R², R³ and R⁴ have the same meaning as defined above and R^{a} may be a C₁₋₂₀ hydrocarbon. Particularly, R^{a} may be a linear, branched or cyclic, saturated or unsaturated C₁-C₁₆ hydrocarbon group. Particularly, R^{a} may be a linear or branched C₁ to C₄ alkyl or alkenyl group, or a cyclic C₃ to C₇ alkyl or alkenyl group, or a phenyl group, optionally substituted with a C₁ to C₄ alkyl group, even more particularly R^{a} may be a methyl group or a phenyl group, most particularly R^{a} may be a phenyl group.

According to a particular embodiment, the properfume compound may be in the form of an alpha-ketoester of formula

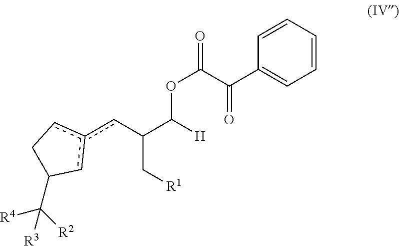

wherein the dotted lines, R¹, R², R³ and R⁴ have the same meaning as defined above.

The properfume compound in the form of an alpha-ketoester releases the invention's compound upon exposure to light.

According another particular embodiment, the properfume compound may be in the form of a heterocycle such as an aminal, imidazolidinone or oxazolidine. Such a properfume may be obtained by the reaction between the invention's compound of formula (I) and a diamine, an amino acid amide, an aminoalkanol or an aminoalkanediol. The person skilled in the art is well aware of methods allowing the preparation of an aminal, imidazolidone or oxazolidine starting from an aldehyde compound of formula (I).

According a particular embodiment, the properfume compound may be in the form of an oxazolidine of formula

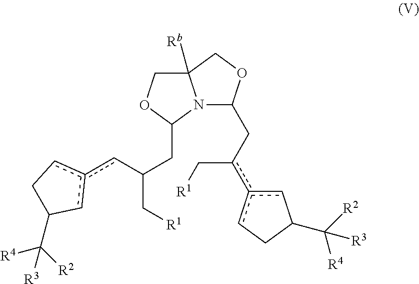

wherein the dotted lines, R¹, R², R³ and R⁴ have the same meaning as defined above and R^{b} may be a hydrogen atom, a methyl group or an ethyl group.

According a particular embodiment, the properfume compound may be in the form of an imidazolidinone of formula

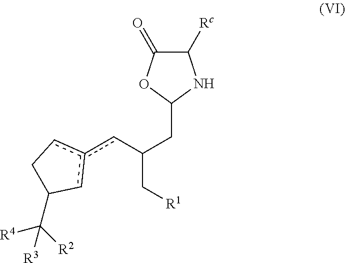

wherein the dotted lines, R¹, R², R³ and R⁴ have the same meaning as defined above and R^{c} may be a hydrogen atom, a C₁ to C₄ alkyl group or a benzyl group. Particularly, R^{c} may be a hydrogen atom, a methyl group or an ethyl group.

The invention's compound is released through hydrolysis of the corresponding aminal, imidazolidinone or oxazolidine.

According another particular embodiment, the properfume compound may be in the form of an enol ether. Such a properfume may be obtained by the reaction between the invention's compound of formula (I), optionally in the form in a form of an acetal and an alcohol. The person skilled in the art is well aware of methods allowing the preparation of an enol ether starting from an aldehyde compound of formula (I).

According a particular embodiment, the properfume compound may be in the form of an enol ether of formula

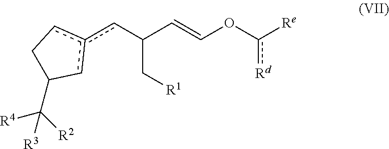

wherein the dotted lines, R¹, R², R³ and R⁴ have the same meaning as defined above and R^{d} may be a hydrogen or a C₁₋₅ alkyl group; Re represents a C₁₋₁₀ alkyl, C₂₋₁₀ alkenyl, C₃₋₁₅ cycloalkyl or C₅₋₁₁ cycloalkenyl group, each optionally substituted with one or more of a C₁₋₅ alkyl, C₁₋₅ alkoxy, C₃₋₈ cycloalkyl, C₅₋₈ cycloalkenyl, C₆ aryl and/or C₆ aryloxy group, each optionally substituted with one or more of a C₁₋₅ alkyl or C₁₋₅ alkoxy group; or R^{c} and R^{d}, when taken together, may form a C₃₋₁₂ cycloalkyl, C₅₋₁₁ cycloalkenyl or C₆₋₁₀ aryl group, each optionally substituted with one or more of a C₁₋₅ alkyl, C₁₋₅ alkoxy, C₃₋₇ cycloalkyl, C₅₋₇ cycloalkenyl and/or C₆ aryl group, each optionally substituted with one or more of a C₁₋₄ alkyl or C₁₋₄ alkoxy group.

The invention's compound is released through oxidation of the corresponding enol ether.

According another particular embodiment, the properfume compound may be in the form of Knoevenagel adduct. Such a properfume may be obtained by the reaction between the invention's compound of formula (I) and ethyl 3-oxobutanoate. The person skilled in the art is well aware of methods allowing the preparation of a Knoevenagel adduct starting from an aldehyde compound of formula (I).

According a particular embodiment, the properfume compound may be in the form of a Knoevenagel adduct of formula

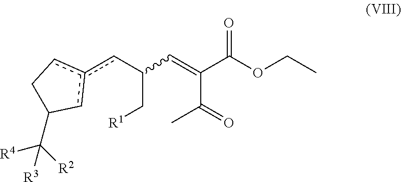

wherein the dotted lines, R¹, R², R³ and R⁴ have the same meaning as defined above.

The invention's compound is released through oxidation of the corresponding Knoevenagel adduct.

According a particular embodiment, the properfume compound may be in the form of an alpha-ketoester of formula (IV′) as defined above or in a form of an enolether of formula (VII) as defined above.

Another object of the present invention is a method for arthropod, preferably insect, control which comprises bringing an arthropod, preferably insect, into direct contact or in contact with vapors of a compound of formula (I) as defined above. In other words, the present invention is a method to confer, enhance, improve or modify the arthropod control properties of an arthropod control composition or of a arthropod control article, which method comprises adding to said composition or article an effective amount of at least a compound of formula (I) as defined above

A further object of the present invention is use of a compound of formula (I) as defined above to control arthropods, preferably insects.

The term “arthropod” has the normal meaning for a skilled person in the technical field. Arthropods include invertebrate animals, such as insects, arachnids, and crustaceans, that have a segmented body and jointed appendages. Arthropods usually have a chitinous exoskeleton molted at intervals, and a dorsal anterior brain connected to a ventral chain of ganglia.

Arthropods in the present invention's understanding relate to undesired arthropods, meaning that their presence in the air, on the surface of an article, the surface of a plant or the surface of a vertebrate, such as a human subject or other mammal, preferably human subject, is not desired. Preferably undesired arthropods are pest arthropods that impact plants and animals, e.g. thrips, aphids, beetles, moth, mealybug, scale etc., more preferably pest arthropods that impact animals, e.g. ants, termites, cockroaches, flies, etc., even more preferably blood feeding arthropods that impact vertebrates, e.g. biting fly, bed bug, kissing bug, flea, lice, mosquitoes and ticks, even more preferably mosquitos and ticks.

The reason why the presence of an arthropod is not desired might be that the arthropod's presence in the air is unpleasant to a subject, the contact of an arthropod on an article transfers diseases and/or germs or the arthropod bites an organism and causes itching, the transmission of diseases and/or germs or the arthropod feeding may be the cause for other diseases and/or conditions.

The expression “control”, “arthropod control” or the like has the normal meaning for a skilled person in the technical field. “Controlling”, in the context of the present invention, defines the ability of a compound or an arthropod controlling composition according to the present invention to attract, deter, kill or repel an arthropod, preferably deter or repel an arthropod and even more preferably repel an arthropod.

“Attracting” according to the present invention defines the ability of a compound or an arthropod attractant composition according to the invention to increase or encourage contact or the presence of an arthropod at the arthropod attractant source, such as in the air, on the surface of an article or on the surface of an vertebrate, such as a human subject or other mammal, preferably an article such as a trapping device, the arthropod attractant compound or composition has been applied to.

“Deterring” according to the present invention defines the ability of a compound or an arthropod deterrent composition according to the invention to minimize, reduce, discourage or prevent contact or the presence of an arthropod at the arthropod deterrent source, such as in the air, on the surface of an article or on the surface of an vertebrate, such as a human subject or other mammal, preferably human subject, to which the arthropod deterrent compound or composition has been applied to. Typically, the deterrent effect is shown when used as feeding deterrent hindering a pest from subsequent food intake or oviposition after an initial tasting of the arthropod deterrent compound or composition.

“Killing” according to the present invention defines the ability of a compound or an art report killing composition according to the present invention to kill an arthropod at the arthropod killing source, such as in the air, on the surface of an article or on the surface of an vertebrate, such as a human subject or other mammal, preferably human subject, to which the arthropod killing compound or composition has been applied to. When an arthropod killing composition is applied to a plant, an animal or human subject, it is applied in an amount which is killing to the arthropod but not to the subject.

“Repellency” according to the present invention defines the ability of a compound or an arthropod repellent composition according to the present invention to minimize, reduce, discourage or prevent approach or the presence of an arthropod at the arthropod repellent source, such as in the air, on the surface of an article or on the surface of an vertebrate, such as a human subject or other mammal, preferably human subject, to which the arthropod repellent compound or composition has been applied to.

Co-ingredients which are used in arthropod control formulation can be added to enhance the control efficacy. In particular one may cite ingredients chosen in the group consisting of ethyl 3-(acetyl(butyl)amino)propanoate (IR3535®), N,N-diethyl-3-methylbenzamide (DEET), p-menthane-3,8-diol (PMD), Eucalyptus citriodora oil, Citronella spp. oil, sec-butyl 2-(2-hydroxyethyl)piperidine-1-carboxylate (picaridin), vanillin, Castor oil, Cedarwood oil, Cinnamon oil, Citronella, Citronella oil, Clove oil, Corn oil, Cornmint, Cornmint oil, Cottonseed oil, 4-Allyl-2-methoxyphenol (Eugenol), Garlic oil, (2E)-3,7-Dimethylocta-2,6-dien-1-ol (Geraniol), Geranium oil, Lemongrass oil, Linseed oil, Peppermint, Peppermint oil, 2-Phenylethyl propionate, Rosemary oil, Sesame oil, Soybean oil, Spearmint, Spearmint oil, Thyme oil, Mint, Mint oil, Pepper extract, Wintergreen oil, citronellal, Lavender oil, Lavandula hybrida extract, Lavandin oil, Lemon oil, Margosa extract, Mentha arvensis extract, Metofluthrin, Nonanoic acid, Pyrethrins and Pyrethroids, 2,3,4,5-bis(butyl-2-ene)tetrahydrofurfural (MGK Repellent 11), cineole, cinnamaldehyde, citral, citronellol, coumarin, dibutyl phthalate, diethyl phthalate, dimethyl anthranilate, dimethyl phthalate, ethyl anthranilate, ethyl vanillin, Eucalyptus oil, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxycitronellal, Lime oil, limonene, linalool, methyl anthranilate, myrcene, Neem oil, Sabinene, β-Caryophyllene, (1H-indol-2-yl)acetic acid, anethole, anise oil, basil oil, bay oil, camphor, ethyl salicylate, evergreen oils, pine oil, Tetramethrin, Allethrin, (RS)-α-cyano-3phenoxybenzyl-(1RS)-cis, Cypermethrin, Prallethrin, Acetamiprid, Azadirachtin, Bendiocarb, Bifenthrin, Chlorpyrifos, Deltamethrin, Diazinon, Dichlorvos, fipronil, imidacloprid, Malathion, Margosa extract, Nicotine, Permethrin, Rotenone, S-Methoprene, Spinosad (Spinosyn A), Spinosyn D, Transfluthrin, anisic alcohol, octahydrocoumarin, (+−)-2,5-dimethyl-2-indanmethanol, 4,4A,5,9B-tetrahydro-indeno[1,2-D]-1,3-dioxin, 2,4-dimethyl-4,4a,5,9b-tetrahydroindeno[1,2-d][1,3]dioxine, and mixtures thereof.

As shown in the examples below, compounds of formula (I) presented herein demonstrate an antimicrobial effect. In some aspects, the antimicrobial effect is bactericidal, wherein bacterial cells are inactivated or killed. Alternatively, in some aspects, the antimicrobial effect is bacteriostatic, wherein the growth rate of bacterial cells is reduced, arrested, or inhibited

Referring to the examples below, the antimicrobial effect of the compounds of formula (I) was discovered by testing a wide range of concentrations (doses) of compounds and observing the bactericidal effect against the noted microbial strains (Staphylococcus aureus and Cutibacterium acnes)

Hence a further aspect of the invention provides an antimicrobial composition for use in a perfuming consumer product comprising one or more compounds of formula (I) as defined above, in particular, 3-(3-isopropylcyclopent-1-en-1-yl)-2-methylpropanal, 3-(4-isopropylcyclopent-1-en-1-yl)-2-methylpropanal or a mixture thereof.

A further aspect of the invention provides the use of composition comprising one or more compounds of formula (I) as an antimicrobial composition. An additional aspect of the invention is wherein the substrate is treated with the composition in an amount sufficient to provide an antimicrobial effect.

By the term “antimicrobial”, it is meant the normal meaning in the art; i.e. the composition kills microorganism or inhibits their growth.

The use of the antimicrobial composition as defined here-in is particularly advantageous to inactivate microorganisms such as bacteria. The antimicrobial effect is one of the main requirements of hygiene products such as body care or home care products. The antimicrobial effect of the compositions provided herein may be determined using any method readily selected by one of ordinary skill in the art. One example of a method for determining the antimicrobial effect of the compositions provided herein is the provided in the examples below.

In some aspects, the composition provides the antimicrobial effect by inhibiting the growth of bacteria. In one aspect, the antimicrobial effect is an inhibition of growth of a bacterial species selected from the group consisting of: Staphylococcus aureus and Cutibacterium acnes.

In some aspects, the composition used in the present disclosure one or more compounds of formula (I) in an amount above 50 ppm of the overall composition. Preferably the one or more compounds of formula (I) does not exceed 100,000 ppm of the overall composition, preferably 50,000 ppm.

Where the bacterial species is Staphylococcus aureus, preferably the compound(s) of formula (I) is present in an amount above 222 ppm, preferably above 333 ppm.

Where the bacterial species is Cutibacterium acnes, preferably the compound(s) of formula (I) is present in an amount above 99 ppm, preferably above 148 ppm.

In some aspects, the present disclosure provides a non-therapeutic method of reducing, arresting or inhibiting the growth rate of bacterial cells or inactivating or killing bacterial cells, the method comprising treating a substrate comprising microbes with an effective amount of at least a compound of formula (I) as defined above

A further object of the present invention provides a method to reduce, arrest, or inhibit the growth rate of bacterial cells or to inactivate or kill bacterial cells which method comprises adding an effective amount of at least a compound of formula (I) as defined above to a composition or consumer product, or contacting or treating the surface with an effective amount of at least a compound of formula (I) as defined above.

In some aspects, the present disclosure provides a perfuming composition comprising:

a composition comprising compound(s) of formula (I) wherein the composition is present in an amount sufficient to provide an antimicrobial effect;

at least one ingredient selected from the group consisting of a perfumery carrier and a perfumery base; and

optionally at least one perfumery adjuvant.

An embodiment of the perfuming composition of the invention is wherein composition is present in an amount sufficient to provide an antimicrobial effect.

In some embodiments, the amount sufficient to provide an antimicrobial effect has no impact on the overall odor profile of the composition.

In some aspects, the present disclosure provides the use or a method of using a composition comprising compound(s) of formula (I) for the preparation of an antimicrobially active consumer product.

The invention's compounds can be prepared according to standard method known in the art as described herein-below.

### EXAMPLES

The invention will now be described in further detail by way of the following examples, wherein the abbreviations have the usual meaning in the art, the temperatures are indicated in degrees centigrade (° C.). NMR spectra were acquired using either a Bruker Avance II Ultrashield 400 plus operating at 400 MHz, (¹H) and 100 MHz (¹³C) or a Bruker Avance III 500 operating at 500 MHz (¹H) and 125 MHz (¹³C) or a Bruker Avance III 600 cryoprobe operating at 600 MHz (¹H) and 150 MHz (¹³C). Spectra were internally referenced relative to tetramethyl silane 0.0 ppm. ¹H NMR signal shifts are expressed in δ ppm, coupling constants (J) are expressed in Hz with the following multiplicities: s, singlet; d, doublet; t, triplet; q, quartet; m, multiplet; b, broad (indicating unresolved couplings) and were interpreted using Bruker Topspin software. ¹³C NMR data are expressed in chemical shift δ ppm and hybridization from DEPT 90 and DEPT 135 experiments, C, quaternary; CH, methine; CH₂, methylene; CH₃, methyl.

### Example 1

### Synthesis of Compounds of Formula (I)

### Preparation of a Composition of Matter Comprising 3-(3-isopropylcyclopent-1-en-1-yl)-2-methylpropanal, 3-(4-isopropylcyclopent-1-en-1-yl)-2-methylpropanal and 3-(3-isopropylcyclopentylidene)-2-methylpropanal

### Step 1: Preparation of 3-i-Propyl-adipic Acid

3-neck 50 mL round bottomed flask equipped with a mag stir bar and reflux condenser was charged with 24 mg of VO₃NH₄ and 28.7 g of 60% HNO₃. The solution was heated to 50° C. then charged dropwise with 9.8 g of 4-i-Pr-cylcyclohexanol. Upon observation of evolution of red gas (NOₓ) with the 1st 0.5 mL of alcohol added, the dosage continued over a 4 h period during which the temperature was maintained between 60-65° C. After the addition was complete, the temperature fell to 50° C. At this point the reaction mixture was heated to 95° C. for 60 min in order to drive off the remaining NOR. The solution was then allowed to slowly cool to 25° C. overnight. The following morning, crystals were observed and the slurry was further cooled with an ice water bath. The white crystals were filtered and dried to give 9.27 g of 98% pure 3-i-Pr-adipic acid.

Yield=69%

¹H-NMR (600 MHz, acetone-d6): δ (ppm) 10.52 (broad singlet, 2H), 2.32 (m, 4H), 2.19 (d, J 10.74 Hz, 1H), 2.17 (d, J 7.14 Hz, 1H), 2.05 (p, J 2.2 Hz, 2H), 1.67-1.3 (m, 4H), 1.51-1.59 (m, 2H), 0.91 (d, J 6.72 Hz, 3H), 0.88 (d, J 6.72 3H)

¹³C-NMR (150 MHz, acetone-d6): δ (ppm) 18.9, 19.5, 27.0, 30.6, 32.2, 35.8, 41.1, 174.76, 174.84

### Step 2: Preparation of 3-i-propylcyclopentanone

100 mL cylindrical flask was charged with 12.5 g of 3-i-Pr-adipic acid and 0.57 g of Ba(OH)₂. The reaction mixture under N₂ atmosphere was heated in a kuegel-rohr oven set to 275° C. during which time water, 3-i-Pr-cyclopentanone and some unreacted 3-i-Pr-adipic acid was distilled off into two other bulbs connected to the reaction flask. The reaction mixture was heated until no liquid remained in the cylindrical flask and the contents turned solid black. After cooling the oven, the collected distillates are weighed giving, 3.29 g of a distilled viscous oil which solidifies upon cooling, composed of 86% pure unreacted 3-i-Pr-adipic acid and 11% 3-i-Pr-cyclopentanone and a liquid fraction composed of 6.33 g of wet 3-i-Pr-cyclopentanone. The latter is diluted in cyclohexane and dried over Na₂SO₄ and then concentrated on the rotovap to give 5.46 g of 3-i-Pr-cyclopentanone at 99% purity. Yield: 91% based on 79% conversion of 3-i-Pr-adipic acid

¹H-NMR (600 MHz, CDCl₃): δ (ppm) 2.28-2.42 (m, 2H), 2.10-2.19 8 m, 2H), 1.79-1.89 (m, 2H), 1.45-1.56 (m, 2H), 0.96 (d, J 6.6 Hz, 3H), 0.93 (d, J 6.6 Hz, 3H)

¹³C-NMR (150 MHz, CDCl₃): δ (ppm) 20.4, 21.3, 27.8, 33.6, 39.2, 43.8, 44.7, 220.0

### Step 3: Preparation of 3-i-propylcyclopentanol

750 mL 3-neck flat bottomed flask attached to a mechanical stirrer was placed under N₂ atmosphere and charged with 15 g of LiAlH₄ pellets, 400 mL of THF and cooled to 0° C. 100 g of the ketone obtained in step 2 was added over a 1.5 h period causing the temperature to rise to a max of 12° C. After stirring for another 30 min, 15 mL of water was carefully added, followed by 15 mL of 5% NaOH, 45 mL of cold water, then further diluted with 100 mL of THF to give a grey-white slurry. The slurry was filtered over a frit containing celite and the filtrate was concentrated on the rotovap to give 104.6 g of concentrate. The latter was charged with 10.07 g of Primol and distilled to give 98 g of clear white distillate containing 99% pure alcohol as a 63/36 mixture of two 3-i-Pr-cyclopentanol diastereomers. Yield=96%

¹H-NMR (600 MHz, CDCl₃): δ (ppm) 4.31-4.35 (m, 1H, minor isomer), 4.24-4.30 (m, 1H, major isomer), 2.13 (p, J 7.08 Hz, 1H), 1.67-1.99 (m, 1H), 1.31-1.63 (m, 5H), 1.11-1.21 (m, 2H), 0.83-0.93 (m, 6H)

¹³C NMR (150 MHz, CDCl₃): δ (ppm) major isomer 21.3, 21.5, 28.3, 33.9, 35.6, 40.7, 46.2, 73.8

minor isomer 21.4, 21.4, 28.6, 33.5, 35.5, 40.7, 45.0, 74.0

### Step 4: Preparation of 7-isopropyl-1-oxaspiro[4.4]nonan-2-one

250 mL 3-neck round bottomed flask equipped with a Dean-Stark trap was charged with 96 g of 3-i-propylcyclopentanol and heated in an oil bath to a pot temp of 152° C. Upon reaching the desired temperature, 14 g of DTBP dissolved in 47 g of butyl acrylate was added over a 3 h 15 min period. After the addition was completed, the yellow reaction mixture was stirred for an additional 15 min, before turning off the heat and the reaction was allowed to cool to 50° C. Upon reaching 50° C., 75 g of 30% NaOH along with 50 mL of MTBE were added to the reaction mixture and stirred at 45-50° C. for 1.5 h. The contents were then transferred to a 1 L separatory funnel, and the reaction flask was rinsed with 75 mL of water and 75 mL of MTBE. After phase separation, the aqueous phase was extracted with 3×200 mL of MTBE, combined, washed with water, dried over Na₂SO₄ and concentrated on the rotovap to give 71.2 g of crude unreacted 3-i-Pr-cyclopentanol. The orange alkaline aqueous phase from above was slowly acidified with 130 g of 50% H₂SO₄, turning it cloudy light yellow. The acidic material was extracted 3×150 mL of ether, combined, dried over Na₂SO₄ and concentrated on the rotovap to give 63.7 g of crude lactone. The latter was distilled under vacuum to give 37.2 g of 98% pure 7-isopropyl-1-oxaspiro[4.4]nonan-2-one (54% yield) as a 1:1 mixture of 2 diastereomers.

¹H-NMR (600 MHz, CDCl₃): δ (ppm) 2.57 (t, J 8.1 Hz, 2H), 1.88-2.21 (m, 5H), 1.71-1.86 (m, 2H), 1.60-1.69 (m, 1H), 1.45-1.55 (m, 1H), 1.27-1.44 (m, 1H), 0.85-0.92 (m, 6H)

¹³C NMR (150 MHz, CDCl₃): δ (ppm) 176.85, 176.83, 95.12, 94.32, 46.19, 45.83, 43.78, 42.92, 38.95, 37.97, 33.95, 33.46, 33.43, 32.84, 29.83, 29.73, 29.315, 28.63, 21.48, 21.23, 21.15, 21.04

### Step 5: Preparation of a Mixture Comprising butyl 3-(3-isopropylcyclopent-1-en-1-yl)propanoate, butyl 3-(4-isopropylcyclopent-1-en-1-yl)propanoate and butyl 3-(3-isopropylcyclopentylidene)propanoates

4-neck 500 mL flat bottomed flask equipped with a magnetic stir bar and Dean-Stark trap was charged with 215 g of n-Butanol and 0.5 g of 96% H₂SO₄. The solution was heated to reflux under N₂ atmosphere and dosed with 60 g of 7-isopropyl-1-oxaspiro[4.4]nonan-2-one over a 3.5 h period. Reflux continued for a total of 9.5 h until 2% of starting material remained. The reaction mixture transferred to a 1 L separatory funnel, washed with saturated NaHCO₃, extracted 3×125 mL of ether, dried over Na₂SO₄ and concentrated on the rotovap to give 86.31 g of concentrate. The latter was distilled under vacuum to give 69.2 g of a mixture of butyl 3-(3-isopropylcyclopent-1-en-1-yl)propanoate (53%), butyl 3-(4-isopropylcyclopent-1-en-1-yl)propanoate (40%) and butyl 3-(3-isopropylcyclopentylidene)propanoates (6%). Yield for all isomers=91%

¹H-NMR (600 MHz, CDCl₃): δ (ppm) 5.33 (d, J 1.6 Hz, 1H, major isomer), 5.29 (broad s, 1H, minor isomer), 4.07 (t, J 6.7 Hz, 2H), 2.15-2.49 (m, 7H), 1.91-2.03 (m, 1H), 1.56-1.64 (m, 2H), 1.32-1.54 (m, 4H), 0.79-0.96 (m, 9H)

¹³C NMR (150 MHz, CDCl₃): δ (ppm) 173.6, 173.57, 143.09, 142.45, 126.98, 123.48, 64.21, 64.20, 52.76, 46.14, 39.54, 36.90, 34.89, 33.55, 32.95, 32.81, 30.71, 27.72, 26.66, 26.62, 21.02, 20.9, 20.46, 20.26, 19.16, 19.15, 13.71

### Step 6: Preparation of a Mixture Comprising butyl 3-isopropylcyclopentenyl-2-methylpropanoates

100 mL 3-neck round bottomed flask equipped with a magnetic stir bar and reflux condenser was charged with 3.4 g of diisopropyl amine, 50 mL of THF and cooled to −60° C. under N₂ atmosphere. 12.2 mL of 2.5M solution of n-BuLi was then added dropwise causing the temperature to rise to −50° C. After stirring for 5 min, 5 g of a mixture of butyl 3-isopropylcyclopentenyl propanoates obtained in step 5 was added over a 7 min period causing the temperature to rise from −60 to −55° C. The yellow solution was stirred for 1.5 h at −70° C. then charged with 10 g of CH₃I over a 1 h period causing the temperature to rise to −60° C. The reaction temperature was allowed to reach 25° C. then stirred overnight for a total of 23 h. The reaction was quenched with saturated NH₄Cl, extracted with ether (3×50 mL), washed with 15% NaHSO₃, dried over Na₂SO₄ and rotovapped to give 5.13 g of concentrate. The latter was distilled on the kuegel-rohr to give 4.38 g of yellow distillate containing a complex mixture of 94% of butyl 3-isopropylcyclopentenyl-2-methylpropanoates (80% yield)

¹H-NMR (600 MHz, CDCl₃): δ (ppm) 5.35 (broad s, 1H, major isomer), 5.31 (broad s, 1H, minor isomer), 4.05 (t, J 6.6 Hz, 2H), 2.61 (p, J 7.2 Hz, 1H), 2.09-2.49 (m, 6H), 1.88-2.02 (m, 2H), 1.10-1.64 (m, 7H), 0.8-0.98 (m, 9H)

¹³C NMR (150 MHz, CDCl₃): δ (ppm) 176.74, 176.71, 141.88, 141.87, 141.28, 141.27, 128.78, 128.74, 125.24, 65.87, 64.12, 64.11, 64.09, 52.76, 52.73, 46.30, 46.20, 39.28, 38.38, 38.27, 38.24, 36.91, 36.88, 35.60, 35.53, 35.50, 34.66, 34.59, 33.54, 32.92, 32.87, 30.74, 30.73, 30.71, 27.79, 27.67, 21.05, 21.02, 20.92, 20.50, 20.43, 20.30, 20.28, 19.17, 17.18, 17.13, 17.10, 15.28, 13.73

### Step 7: Preparation of a Mixture Comprising 3-(3-isopropylcyclopent-1-en-1-yl)-2-methylpropanal, 3-(4-isopropylcyclopent-1-en-1-yl)-2-methylpropanal and 3-(3-isopropylcyclopentylidene)-2-methylpropanal

750 mL 4-neck flat bottomed flask equipped with a magnetic stir bar and reflux condenser was charged with 26 g of butyl 3-isopropylcyclopentenyl-2-methylpropanoates obtained in step 6, 300 mL of CH₂Cl₂ and cooled to −78° C. 98 mL of 1.2M DIBALH in toluene was then added over a 1.5 h period. At the end of the DIBALH addition, the reaction was quenched at −72° C. with the addition of 230 g of 10% NaK Tartrate. The mixture was stirred for 2 h at 25° C. then transferred to a separatory funnel. After phase separation, the aqueous phase was extracted 3×200 mL with CH₂Cl₂, combined, washed with saturated NaCl, dried over Na₂SO₄ and rotovapped to give 21.46 g of concentrate. The crude mixture of 3-isopropylcyclopent-1-enyl-2-methylpropanal was chromatographed with silica using 94/6 pentane/ether as the solvent to give 12.8 g of a mixture of 3-(3-isopropylcyclopent-1-en-1-yl)-2-methylpropanal (53%), 3-(4-isopropylcyclopent-1-en-1-yl)-2-methylpropanal (39%) and 3-(3-isopropylcyclopentylidene)-2-methylpropanal (6%). Yield for all isomers 74% yield.

¹H-NMR (600 MHz, CDCl₃): δ (ppm) 9.63 (broad d, 1H, major regioisomer), 9.62 (broad d, 1H, minor regioisomer), 5.37 (broad s, 1H, major regioisomer), 5.34 (broad s, 1H, minor regioisomer), 2.06-2.58 (m, 3H), 1.9-2.05 (m, 2H), 1.43-1.55 (m, 2H), 1.02-1.09 (m, 2H), 0.8-0.93 (m, 9H)

¹³C NMR (150 MHz, CDCl₃): δ (ppm) 205.13, 205.11, 141.09, 141.06, 140.48, 129.54, 129.44, 125.95, 52.74, 52.71, 46.26, 46.15, 44.68, 44.62, 44.59, 39.50, 39.46, 36.89, 36.88, 34.86, 34.77, 33.50, 33.49, 32.84, 32.81, 32.56, 32.53, 32.48, 27.69, 27.63, 21.01, 20.99, 20.89, 20.87, 20.46, 20.41, 20.26, 20.24, 13.58, 13.54, 13.52, 13.51

### Preparation a Composition of Matter Comprising 3-(3-ethylcyclopent-1-en-1-yl)-2-methylpropanal, 3-(4-ethylcyclopent-1-en-1-yl)-2-methylpropanal and 3-(3-ethylcyclopentylidene)-2-methylpropanal

Said composition of matter was prepared according to steps 1 to 7 reported for the preparation of a composition of matter comprising 3-(3-isopropylcyclopent-1-en-1-yl)-2-methylpropanal, 3-(4-isopropylcyclopent-1-en-1-yl)-2-methylpropanal and 3-(3-isopropylcyclopentylidene)-2-methylpropanal and therefore started with 4-ethylcyclohexanol. Overall yield=11%

¹H-NMR (600 MHz, CDCl₃): δ (ppm) 9.636 (broad s, 1H, diastereomer of minor regioisomer), 9.633 (broad s, 1H, diastereomer of major regioisomer), 9.629 (broad s, 1H, diastereomer of major regioisomer), 9.625 (broad s, 1H, diastereomer of minor regioisomer), 5.36 (broad dd, 1H, minor regioisomer), 5.33 (broad d, 1H, major regioisomer), 1.82-2.60 (m, 6H), 1.23-1.47 (m, 2H), 1.03-1.11 (m, 2H), 0.82-0.94 (m, 6H)

¹³C NMR (150 MHz, CDCl₃): δ (ppm) 205.15, 205.12, 205.10, 140.54, 140.50, 140.14, 140.13, 130.96, 130.88, 125.61, 125.58, 47.24, 47.22, 44.67, 44.64, 44.62, 41.22, 41.19, 40.03, 39.95, 38.59, 38.58, 34.61, 34.54, 32.53, 32.48, 32.45, 32.40, 29.93, 29.91, 29.35, 29.33, 28.85, 13.57, 13.54, 13.49, 12.56, 12.55, 12.07, 12.04

### Preparation of a Composition of Matter Comprising 3-(3-n-propylcyclopent-1-en yl)-2-methylpropanal, 3-(4-n-propylcyclopent-1-en-1-yl)-2-methylpropanal and 3-(3-n-propylcyclopentylidene)-2-methylpropanal

Said composition of matter was prepared according to steps 1 to 7 reported for the preparation of a composition of matter comprising 3-(3-isopropylcyclopent-1-en-1-yl)-2-methylpropanal, 3-(4-isopropylcyclopent-1-en-1-yl)-2-methylpropanal and 3-(3-isopropylcyclopentylidene)-2-methylpropanal and therefore started with 4-n-propylcyclohexanol. Overall yield=3%

¹H-NMR (600 MHz, CDCl₃): δ (ppm) 9.61-9.64 (overlapping doublets, 1H), 5.35 (broad d, 1H, major regioisomer), 5.33 (broad dd, 1H, minor regioisomer), 1.83-2.66 (m, 8H), 1.17-1.47 (m, 4H), 1.07 (d, J 6.9 Hz, 3H, major regioisomer), 0.89 (t, J 7.26 Hz, 3H, major regioisomer)

¹³C NMR (150 MHz, CDCl₃): δ (ppm) 205.14, 140.30, 131.24, 131.17, 125.62, 125.59, 45.32, 44.67, 44.64, 44.62, 41.54, 41.50, 38.96, 38.92, 38.90, 38.88, 38.55, 38.53, 38.05, 37.97, 34.56, 34.48, 32.53, 32.49, 32.44, 32.40, 30.40, 30.39, 21.33, 21.32, 20.99, 20.98, 14.33, 14.33, 14.25, 13.57, 13.53, 13.50, 13.49

### Preparation of a Composition of Matter Comprising 3-(3-n-butylcyclopent-1-en-1-yl)-2-methylpropanal, 3-(4-n-butylcyclopent-1-en-1-yl)-2-methylpropanal and 3-(3-n-butylcyclopentylidene)-2-methylpropanal

Said composition of matter was prepared according to steps 1 to 7 reported for the preparation of a composition of matter comprising 3-(3-isopropylcyclopent-1-en-1-yl)-2-methylpropanal, 3-(4-isopropylcyclopent-1-en-1-yl)-2-methylpropanal and 3-(3-isopropylcyclopentylidene)-2-methylpropanal and therefore started with 4-n-butylcyclohexanol. Overall yield=9%

¹H-NMR (600 MHz, CDCl₃): δ (ppm)) 9.63 (broad d, 1H), 5.35 (broad d, 1H, major regioisomer), 5.32 (broad dd, 1H, minor regioisomer), 1.83-2.63 (m, 9H), 1.18-1.46 (m, 5H), 1.07 (d, J 6.9 Hz, 3H, major regioisomer), 0.88 (t, J 7.26 Hz, 3H, major regioisomer)

¹³C NMR (150 MHz, CDCl₃): δ (ppm) 205.14, 140.35, 140.29, 140.18, 140.16, 131.28, 131.21, 125.62, 125.60, 76.81, 45.56, 45.55, 44.67, 44.64, 44.62, 41.58, 41.54, 38.94, 38.93, 38.30, 38.22, 36.38, 36.34, 35.96, 35.95, 34.57, 34.49, 32.53, 32.49, 32.45, 32.40, 30.52, 30.51, 30.43, 30.42, 30.14, 30.12, 22.92, 22.86, 14.14, 14.13, 13.57, 13.54, 13.50, 13.49

### Preparation of a Composition of Matter Comprising 3-(3-tert-butylcyclopent-1-en-1-yl)-2-methylpropanal, 3-(4-tert-butylcyclopent-1-en-1-yl)-2-methylpropanal and 3-(3-tert-butylcyclopentylidene)-2-methylpropanal

Said composition of matter was prepared according to steps 1 to 7 reported for the preparation of a composition of matter comprising 3-(3-isopropylcyclopent-1-en-1-yl)-2-methylpropanal, 3-(4-isopropylcyclopent-1-en-1-yl)-2-methylpropanal and 3-(3-isopropylcyclopentylidene)-2-methylpropanal and therefore started with 4-tert-butylcyclohexanol (prepared according to JAGS 1939, 61, 2728). Overall yield=11%

¹H-NMR (600 MHz, CDCl₃): δ (ppm) 9.64 (d, J 1.86 Hz 1H, major regioisomer), 9.630 (d, J 1.2 Hz 1H, minor regioisomer, diastereomer A), 9.627 (d, J 1.2 Hz 1H, minor regioisomer, diastereomer B), 5.37 (overlapping d, 1H, major regioisomer), 5.32 (broad dd, 1H, minor regioisomer), 2.41-2.58 (m, 4H), 2.00-2.28 (m, 2H), 1.81-1.93 (m, 1H), 1.56-1.66 (m, 1H), 1.07 (d, J 6.78 Hz, 3H), 0.84 (s, 9H, t-butyl group, minor regioisomer), 0.824 (s, 9H, t-butyl group, major regioisomer, diastereomer A), 0.821 (s, 9H, t-butyl group, major regioisomer, diastereomer B)

¹³C NMR (150 MHz, CDCl₃): δ (ppm) 205.11, 205.09, 141.47, 141.43, 128.75, 128.56, 125.84, 125.81, 56.87, 56.85, 49.48, 49.35, 44.70, 44.62, 44.61, 44.57, 36.57, 36.51, 35.05, 34.92, 33.95, 33.18, 33.17, 32.59, 32.58, 32.51, 32.24, 32.22, 27.42, 27.39, 27.37, 27.36, 25.54, 25.53, 13.58, 13.54, 13.49

### Preparation of a Composition of Matter Comprising 3-(3-sec-butylcyclopent-1-en-1-yl)-2-methylpropanal, 3-(4-sec-butylcyclopent-1-en-1-yl)-2-methylpropanal and 3-(3-sec-butylcyclopentylidene)-2-methylpropanal

Said mixtures was prepared according to steps 1 to 7 reported for the preparation of a composition of matter comprising 3-(3-isopropylcyclopent-1-en-1-yl)-2-methylpropanal, 3-(4-isopropylcyclopent-1-en-1-yl)-2-methylpropanal and 3-(3-isopropylcyclopentylidene)-2-methylpropanal and therefore started with 4-sec-butylcyclohexanol. Overall yield=1%

¹H-NMR (600 MHz, CDCl₃): δ (ppm) 9.63 (d, J 1.86 Hz, 1H, major regioisomer), 9.62 (d, J 1.80 Hz, 1H, minor regioisomer), 5.33-5.37 (broad s, 1H), 1.87-2.64 (m, 5H), 1.20-56 (m, 3H), 1.03-1.15 (m, 3H), 0.76-0.90 (m, 9H)

¹³C NMR (150 MHz, CDCl₃): δ (ppm) 205.13, 141.12, 141.10, 140.81, 140.76, 140.47, 130.00, 129.88, 129.18, 129.09, 125.95, 125.94, 125.93, 125.92, 51.09, 51.07, 50.80, 50.79, 44.69, 44.63, 44.62, 44.60, 44.31, 44.29, 44.2, 44.18, 39.85, 39.83, 39.81, 39.67, 39.63, 39.55, 39, 38.98, 38.97, 38.91, 37.06, 36.37, 36.36, 34.9, 34.82, 34.81, 34.73, 32.5, 32.53, 32.48, 28.09, 28.06, 27.66, 27.65, 27.55, 27.44, 27.42, 27.3, 27.27, 26.71, 26.64, 16.73, 16.71, 16.62, 16.61, 16.46, 16.42, 16.01, 13.59, 13.55, 13.52, 11.79, 11.77, 11.74, 11.72, 11.38, 11.36, 11.35

### Example 2

### Preparation of a Perfuming Composition

A perfuming composition for fine fragrance was prepared by admixing the following ingredients:

| Ingredient name | Parts by weight |
|---|---|
| 10% * ⅔-methylbutyl acetate | 40 |
| 2-methoxy-4-(1-propenyl)phenyl acetate | 10 |
| Benzyl acetate | 200 |
| (Z)-3-hexenyl acetate | 10 |
| Guaiyl acetate | 10 |
| 1,5-dimethyl-1-vinyl-4-hexenyl acetate | 200 |
| 1-phenylethyl acetate | 40 |
| 1(1R,2S,5S,8R)-7,7-dimethyl-6-methylenetricyclo[6.2.1.0^{1,5}]undec-2- | 10 |
| yl]methyl acetate and (2E)-2-[(8R,8as)-8,8a-dimethyl-3,4,6,7,8,8a- | |
| hexahydro-2(1H)-naphthalenylidene]propyl acetate | |
| 10% * dodecanal | 10 |
| 10% * nonanal | 10 |
| (2E)-2-benzylideneoctanal | 400 |
| (10E)-oxacycloheptadec-10-en-2-one | 60 |
| (−)-(3aR,5aS,9aS,9bR)-3a,6,6,9a-tetramethyldodecahydronaphtho[2,1- | 40 |
| b]furan | |
| (+−)-(1E)-1-(2,6,6-trimethyl-½-cyclohexen-1-yl)-1,6-heptadien-3-one | 40 |
| 10% * methyl 2-aminobenzoate | 60 |
| 1% * ethyl 2-methylpentanoate | 40 |
| 1,4-dioxacycloheptadecane-5,17-dione | 200 |
| 10% * 7-methyl-2H-1,5-benzodioxepin-3(4H)-one | 20 |
| 2-methyl-4-phenyl-2-butanol | 100 |
| 2-methyl-4-phenyl-2-butanol | 40 |
| (Z)-3-hexen-1-ol | 10 |
| Citron Smufa oil | 80 |
| (+−)-3,7-dimethyl-6-octen-1-ol | 200 |
| 1% * (2E)-1-(2,6,6-trimethyl-1,3-cyclohexadien-1-yl)-2-buten-1-one | 20 |
| 10% * (2E)-1-(2,6,6-trimethyl-2-cyclohexen-1-yl)-2-buten-1-one | 20 |
| 4-decanolide | 10 |
| 2,6-dimethyl-7-octen-2-ol | 40 |
| 2-Benzyl-1,3-dioxolane-4-methanol and 2-benzyl-1,3-dioxan-5-ol | 80 |
| 3,7-dimethyl-1,6-nonadien-3-ol | 600 |
| Oxacyclohexadec-11-en-2-one | 400 |
| Tetrahydro-2-isobutyl-4-methyl-4(2h)-pyranol | 600 |
| 4-nonanolide | 10 |
| 5-heptyldihydro-2(3H)-furanone | 20 |
| (E)-3,7-dimethyl-2,6-ociadien-1-ol | 40 |
| Geranium rose oil | 20 |
| (1RS,2SR,8RS)-2-(8-isopropyl-6-methyl-bicyclo[2.2.2]oct-5-en-2-yl)- | 100 |
| 1,3-dioxolane | |
| 1-oxa-12/13-cyclohexadecen-2-one | 200 |
| Methyl 2-((1RS,2RS)-3-oxo-2-pentylcyclopentyl)acetate | 2400 |
| Methyl [(1RS,2RS)-3-oxo-2-pentylcyclopentyl]acetate abd methyl | 300 |
| [(1RS,2SR)-3-oxo-2-pentylcyclopentyl]acetate | |
| 3-(1,3-benzodioxol-5-yl)-2-methylpropanal | 400 |
| Benzo[d][1,3]dioxole-5-carbaldehyde | 20 |
| 7-hydroxy-3,7-dimethyloctanal | 200 |
| 10% * indole | 20 |
| 1-(octahydro-2,3,8,8-tetramethyl-2-naphtalenyl)-1-ethanone | 200 |
| (3Z)-hex-3-en-1-yl methyl carbonate | 10 |
| 3,7-dimethyl-1,6-octadien-3-ol | 40 |
| 10% * 6-methoxy-2,6-dimethylheptanal | 20 |
| (E)-3-methyl-4-(2,6,6-trimethyl-2-cyclohexen-1-yl)-3-buten-2-one | 200 |
| 3-methylcyclopentadecanone | 20 |
| 10% * methyl 2-nonynoate | 40 |
| 7-methoxy-3,7-dimethyl-2-octanol | 200 |
| 10% * 4-methyl-2-(2-methyl-1-propen-1-yl)tetrahydro-2H-pyran | 20 |
| 0.1% * (2e,6z)-2,6-nonadienal | 80 |
| 10% * (2-isopropoxyelhyl)benzene | 40 |
| 2-phenylethanol | 100 |
| 3-methyl-5-phenyl-1-pentanol | 400 |
| 3,3-dimethyl-5-(2,2,3-trimethyl-3-cyclopenten-1-yl)-4-penten-2-ol | 60 |
| Cedran-8-yl acetate | 40 |
| Prunella ®¹⁾ | 40 |
| (+−)-2,2,2-trichloro-1-phenylethyl acetate | 20 |
| Benzyl salicylate | 400 |
| (3Z)-3-hexen-1-yl salicylate | 200 |
| Santal album australie he | 80 |
| Alpha-terpineol | 100 |
| 10% * 4-hydroxy-3-methoxybenzaldehyde | 40 |
| 10% * 2,2,5-trimethyl-5-pentylcyclopentanone | 20 |
| Ylang extra | 20 |
| 10% * 2,4-Dimethyl-3-cyclohexene-1-carbaldehyde | 80 |
| | 9800 |

* in dipropyleneglycol
** in isopropyle myristate
¹⁾origin: Firmenich SA, Geneva, Switzerland

The addition of 200 parts by weight of a composition of matter as prepared in example 1 comprising 3-(3-isopropylcyclopent-1-en-1-yl)-2-methylpropanal, 3-(4-isopropylcyclopent-1-en-1-yl)-2-methylpropanal and 3-(3-isopropylcyclopentylidene) methylpropanal to the above-described composition imparted to the latter a floral connotation in the direction of linden blossom while reinforcing green note towards bud.

In addition, the composition acquired a cleaner character. The invention's compound blends particularly well with floral-imparting ingredient of the composition (e.g. (2E)-2-benzylideneoctanal, 3,7-dimethyl-6-octen-1-ol or Ylang extract) and also with green-imparting ingredients of the composition (e.g. (Z)-3-hexenyl acetate, 1-phenylethyl acetate or 2,4-Dimethyl-3-cyclohexene-1-carbaldehyde) and aldehydic-imparting ingredients of the composition (e.g. dodecanal or nonanal).

When instead of the invention's compound, the same amount of 3-[(4S)-4-isopropyl-1-cyclohexen-1-yl]-2-methylpropanal was used, the composition acquired a floral connotation in the direction of Lily of valley allied with watery and fruity-white peach notes. As the invention's compound, 3-[(4S)-4-isopropyl-1-cyclohexen-1-yl]-2-methylpropanal blends particularly well with floral-imparting ingredient of the composition (e.g. (2E)-2-benzylideneoctanal, 3,7-dimethyl-6-octen-1-ol or Ylang extract) however also with watery-imparting ingredients of the composition (e.g. 7-methyl-2H-1,5-benzodioxepin-3(4H)-one) and lactonic-imparting ingredients of the composition (e.g. 4-nonanolide).

### Example 3

### Preparation of an Eau De Toilette Comprising the Invention's Composition of Matter

The eau de toilette was prepared by adding 12% by weight, relative to the total weight of the eau de toilette, of the invention's composition of Example 2 into ethanol.

### Example 4

### Preparation of a Liquid Detergent Comprising the Invention's Compound

TABLE 2
Composition of the liquid detergent formulation

| | Concentration |
|---|---|
| Ingredients | [wt %] |
| Sodium C14-17 Alkyl Sec Sulfonate¹⁾ | 7 |
| Fatty acids, C12-18 and C18-unsaturated²⁾ | 7.5 |
| C12/14 fatty alcohol polyglycol ether with 7 | 17 |
| mol EO³⁾ | |
| Triethanolamine | 7.5 |
| Propylene Glycol | 11 |
| Citric acid | 6.5 |
| Potassium Hydroxyde | 9.5 |
| Properase L⁴⁾ | 0.2 |
| Puradax EG L⁴⁾ | 0.2 |
| Purastar ST L⁴⁾ | 0.2 |
| Acrylates/Steareth-20 Methacrylate | 6 |
| structuring Crosspolymer⁵⁾ | |
| Deionized Water | 27.4 |

¹⁾Hostapur SAS 60; Origin: Clariant
²⁾Edenor K 12-18; Origin: Cognis
³⁾Genapol LA 070; Origin: Clariant
⁴⁾Origin: Genencor International
⁵⁾Aculyn 88; Origin: Dow Chemical

The liquid detergent is prepared by adding 0.5 to 1.5% by weight, relative to the total weight of the liquid detergent, of the invention's perfuming composition of Example 2 into the unperfumed liquid detergent formulation of Table 2 under gentle shaking.

### Example 5

### Preparation of a Fabric Softener Comprising the Invention's Compound

TABLE 3
Composition of the softener formulation

| Ingredient | Concentration [wt %] |
|---|---|
| Methyl bis[ethyl (tallowate)]-2- | 12.20 |
| hydroxyethyl ammonium methyl | |
| sulfate¹⁾ | |
| 1,2-benzisothiazolin-3-one²⁾ | 0.04 |
| CaCl₂ (10% aqueous solution) | 0.40 |
| Water | 87.36 |

¹⁾Stepantex VL90 A Diester Quat; Origin: Stepan
²⁾Proxel GXL; Origin: Arch

The softener is prepared by weighting Methyl bis[ethyl (tallowate)]-2-hydroxyethyl ammonium methyl sulfate which was heated at 65° C. Then Water and 1,2-benzisothiazolin-3-one are placed in the reactor and are heated at 65° C. under stirring. To the above mixture is added Methyl bis[ethyl (tallowate)]-2-hydroxyethyl ammonium methyl sulfate. The mixture is stirred 15 minutes and CaCl₂ is added. Then 0.5 to 2% by weight, relative to the total weight of the softener, of the invention's perfuming composition of Example 2 is added. The mixture is stirred 15 minutes and is cooled down to room temperature under stirring (viscosity measure: result 35+/−5 mPas. (shear rate 106 sec-1)).

### Example 6

### Preparation of a Transparent Isotropic Shampoo Comprising the Invention's Composition

TABLE 4
Composition of the transparent isotropic shampoo formulation

| | | Concentration |
|---|---|---|
| Phases | Ingredients | [wt %] |
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous sol. till pH 5.5-6 | q.s. |

¹⁾ Ucare Polymer JR-400, Origin: Noveon
²⁾ Origin: Schweizerhall
³⁾ Glydant, Origin: Lonza
⁴⁾ Texapon NSO IS, Origin: Cognis
⁵⁾ Tego Betain F 50, Origin: Evonik
⁶⁾ Amphotensid GB 2009, Origin: Zschimmer & Schwarz
⁷⁾ Monomuls 90 L-12, Origin: Gruenau
⁸⁾ Nipagin Monosodium, Origin: NIPA

The shampoo is prepared by dispersed in water Polyquaternium-10. The remaining ingredients of phase A are mixed separately by addition of one after the other while mixing well after each adjunction. This pre-mix is added to the Polyquaternium-10 dispersion and mixed for another 5 min. Then, the premixed phase B and the premixed Phase C are added (Monomuls 90 L-12 is heated to melt in Texapon NSO IS) while agitating. Phase D and Phase E are added while agitating. PH is adjusted with citric acid solution till pH: 5.5-6.0 leading to an unperfumed shampoo formulae.

The perfumed shampoo is prepared by adding 0.4 to 0.8% by weight, relative to the total weight of the shampoo, of the invention's perfuming composition of Example 2 into the unperfumed shampoo formulation of Table 4 under gentle shaking.

### Example 7

### Preparation of a Structured Shower Gel Comprising the Invention's Composition

TABLE 5
Composition of the shower gel formulation

| Ingredients | Amount (% wt) |
|---|---|
| WATER deionised | 49.350 |
| Tetrasodium EDTA ¹⁾ | 0.050 |
| Acrylates Copolymer²⁾ | 6.000 |
| Sodium C12-C15 Pareth Sulfate ³⁾ | 35.000 |
| Sodium Hydroxide 20% aqueous solution | 1.000 |
| Cocamidopropyl Betaine⁴⁾ | 8.000 |
| Methylchloroisothiazolinone and | 0.100 |
| Methylisothiazolinone⁵⁾ | |
| Citric Acid (40%) | 0.500 |

¹⁾ EDETA B POWDER; trademark and origin: BASF
²⁾CARBOPOL AQUA SF-1 POLYMER; trademark and origin: NOVEON
³⁾ ZETESOL AO 328 U; trademark and origin: ZSCHIMMER & SCHWARZ
⁴⁾TEGO-BETAIN F 50; trademark and origin: GOLDSCHMIDT
⁵⁾KATHON CG; trademark and origin: ROHM & HASS

The shower gel is prepared by adding 0.5 to 1.5% by weight, relative to the total weight of the shower gel, of the invention's perfuming composition of Example 2 into the unperfumed shower gel formulation of Table 5 under gentle shaking.

### Example 8

### Preparation of a Transparent Shower Gel Comprising the Invention's Composition

TABLE 6
Composition of the transparent shower gel formulation

| Ingredients | Concentration (% wt) |
|---|---|
| WATER deionized | 52.40 |
| Tetrasodium EDTA ¹⁾ | 0.10 |
| Sodium Benzoate | 0.50 |
| Propylene Glycol | 2.00 |
| Sodium C12-C15 Pareth Sulfate ²⁾ | 35.00 |
| Cocamidopropyl Betaine³⁾ | 8.00 |
| Polyquaternium-7⁴⁾ | 0.20 |
| Citric Acid (40%) | 1.00 |
| Sodium Chloride | 0.80 |

¹⁾ EDETA B POWDER; trademark and origin: BASF
²⁾ ZETESOL AO 328 U; trademark and origin: ZSCHIMMER & SCHWARZ
³⁾TEGO-BETAIN F 50; trademark and origin: GOLDSCHMIDT
⁴⁾MERQUAT 550; trademark and origin: LUBRIZOL

The transparent shower gel is prepared by adding 0.5 to 1.5% by weight, relative to the total weight of the shower gel, of the invention's perfuming composition of Example 2 into the unperfumed shower gel formulation of Table 6 under gentle shaking.

### Example 9

### Preparation of a Milky Shower Gel Comprising the Invention's Composition

TABLE 7
Composition of the milky shower gel formulation

| | Concentration |
|---|---|
| Ingredients | (% wt) |
| WATER deionized | 50.950 |
| Tetrasodium EDTA ¹⁾ | 0.050 |
| Sodium Benzoate | 0.500 |
| Glycerin 86% | 3.500 |
| Sodium Laureth Sulfate ²⁾ | 27.000 |
| Polyquaternium-7³⁾ | 1.000 |
| Coco-Betaine⁴⁾ | 6.000 |
| PEG-120 Methyl Glucose trioleate⁵⁾ | 1.000 |
| Citric Acid (40%) | 1.000 |
| Glycol Distearate & Laureth-4 & Cocamidopropyl Betaine⁶⁾ | 3.000 |
| Sodium Chloride 20% | 5.000 |
| PEG-40 Hydrogenated Castor Oil⁷⁾ | 1.000 |

¹⁾ EDETA B POWDER; trademark and origin: BASF
²⁾ Texapon NSO IS; trademark and origin: COGNIS
³⁾MERQUAT 550; trademark and origin: LUBRIZOL
⁴⁾DEHYTON AB-30; trademark and origin: COGNIS
⁵⁾GLUCAMATE LT; trademark and origin: LUBRIZOL
⁶⁾EUPERLAN PK 3000 AM; trademark and origin: COGNIS
⁷⁾CREMOPHOR RH 40; trademark and origin: BASF

The transparent shower gel is prepared by adding 0.5 to 1.5% by weight, relative to the total weight of the shower gel, of the invention's perfuming composition of Example 2 into the unperfumed shower gel formulation of Table 7 under gentle shaking.

### Example 10

### Preparation of a Pearly Shampoo Comprising the Invention's Composition

TABLE 8
Composition of the pearly isotropic shampoo formulation

| | | Concentration |
|---|---|---|
| Phases | Ingredients | (% wt) |
| A | Water deionized | 45.97 |
| | Tetrasodium EDTA ¹⁾ | 0.05 |
| | Guar Hydroxypropyltrimonium Chloride ²⁾ | 0.05 |
| | Polyquaternium-10 ³⁾ | 0.075 |
| B | NaOH 10% aqueous sol. | 0.3 |
| C | Ammonium Lauryl Sulfate ⁴⁾ | 34 |
| | Ammonium Laureth Sulfate ⁵⁾ | 9.25 |
| | Cocamidopropyl Betaine ⁶⁾ | 2 |
| | Dimethicone (&) C12-13 Pareth-4 (&) | 2.5 |
| | C12-13 Pareth-23 (&) Salicylic Acid ⁷⁾ | |
| D | Cetyl Alcohol ⁸⁾ | 1.2 |
| | Cocamide MEA ⁹⁾ | 1.5 |
| | Glycol Distearate ¹⁰⁾ | 2 |
| E | Methylchloroisothiazolinone & | 0.1 |
| | Methylisothiazolinone ¹¹⁾ | |
| | D-Panthenol 75% ¹²⁾ | 0.1 |
| | Water deionized | 0.3 |
| F | Sodium Chloride 25% aqueous sol. | 0.6 |

¹⁾ EDETA B Powder, Origin: BASF
²⁾ Jaguar C14 S, Origin: Rhodia
³⁾ Ucare Polymer JR-400, Origin: Noveon
⁴⁾ Sulfetal LA B-E, Origin: Zschimmer & Schwarz
⁵⁾ Zetesol LA, Origin: Zschimmer & Schwarz
⁶⁾ Tego Betain F 50, Origin: Evonik
⁷⁾ Xiameter MEM-1691, Origin: Dow Corning
⁸⁾ Lanette 16, Origin: BASF
⁹⁾ Comperlan 100, Origin: Cognis
¹⁰⁾ Cutina AGS, Origin: Cognis
¹¹⁾ Kathon CG, Origin: Rohm & Haas
¹²⁾ D-Panthenol, Origin: Roche

The shampoo is prepared by dispersed in water and Tetrasodium EDTA, Guar Hydroxypropyltrimonium Chloride and Polyquaternium-10. NaOH 10% solution (Phase B) is added once Phase A is homogeneous. Then, the premixed Phase C is added. and mixture is heated to 75° C. Phase D ingredients are added and mixed till homogeneous. The mixture is cooled down. At 45° C., Phase E ingredients are added while mixing. Final viscosity is adjusted with 25% NaCl solution and pH of 5.5-6 is adjusted with 10% NaOH solution.

The perfumed pearly shampoo is prepared by adding 0.4 to 0.8% by weight, relative to the total weight of the shampoo, of the invention's perfuming composition of Example 2 into the unperfumed shampoo formulation of Table 8 under gentle shaking.

### Example 11

### Preparation of a Structured Shower Gel Comprising the Invention's Composition

TABLE 9
Composition of the milky shower gel formulation

| Ingredients | Amount (% wt) |
|---|---|
| WATER deionised | 49.350 |
| Tetrasodium EDTA ¹⁾ | 0.050 |
| Acrylates Copolymer²⁾ | 6.000 |
| Sodium C12-C15 Pareth Sulfate ³⁾ | 35.000 |
| Sodium Hydroxide 20% aqueous solution | 1.000 |
| Cocamidopropyl Betaine⁴⁾ | 8.000 |
| Methylchloroisothiazolinone and Methylisothiazolinone⁵⁾ | 0.100 |
| Citric Acid (40%) | 0.500 |

⁶⁾ EDETA B POWDER; trademark and origin: BASF
⁷⁾ CARBOPOL AQUA SF-1 POLYMER; trademark and origin: NOVEON
⁸⁾ ZETESOL AO 328 U; trademark and origin: ZSCHIMMER & SCHWARZ
⁹⁾ TEGO-BETAIN F 50; trademark and origin: GOLDSCHMIDT
¹⁰⁾ KATHON CG; tradeark and origin: ROHM & HASS

The transparent shower gel is prepared by adding 0.5 to 1.5% by weight, relative to the total weight of the shower gel, of the invention's perfuming composition of Example 2 into the unperfumed shower gel formulation of Table 9 under gentle shaking.

### Example 12

### Preparation of Anhydrous Antiperspirant Spray Formulations Comprising the Invention's Composition

TABLE 10
Composition of the anhydrous antiperspirant spray formulation

| Ingredients | Amount (% wt) |
|---|---|
| Cyclomethicone ⁽¹⁾ | 53.0 |
| Isopropyl myristate | 9.0 |
| Silica ⁽²⁾ | 1.0 |
| Quaternium-18-hectorite ⁽³⁾ | 3.3 |
| Aluminium chlorohydrate ⁽⁴⁾ | 32.7 |
| Perfume oil | 1 |

⁽¹⁾ Dow Corning ® 345 Fluid; origin: Dow Corning
⁽²⁾ Aerosil ® 200; origin: Evonik
⁽³⁾ Bentone ® 38; origin: Elementis Specialities
⁽⁴⁾ Micro Dry Ultrafine; origin: Reheis

Anhydrous antiperspirant spray formulation is prepared by using a high speed stirrer. Silica and Quaternium-18-hectorite are added to the mixture of isopropyl myristate and cyclomethicone. Once completely swollen, aluminium chlorohydrate is added portion-wise under stirring until the mixture becomes homogeneous and without lumps. Then a perfume oil being the invention's perfuming composition of Example 2 is added.

### Example 13

### Preparation of Deodorant Spray Emulsion Formulations Comprising the Invention's Composition

TABLE 11
Composition of deodorant spray emulsion formulation

| Ingredients | Amount (% wt) |
|---|---|
| Ethanol (95%) | 89.25 |
| Triclosan ⁽¹⁾ | 0.25 |
| Isopropyl myristate | 9.00 |
| Invention’s perfuming composition of Example 2 | 1.5 |

⁽¹⁾ Irgasan ® DP 300; origin: BASF
⁽²⁾ mixture of Compounds 2a/2b ca. 45:55

Deodorant spray emulsion formulation is prepared by mixing and dissolving all the ingredients according to the sequence of Table 11. Aerosol cans are filled, and the propellant is crimped and added. Aerosol filling: 40% active solution 60% propane/butane (2.5 bar).

### Example 14

### Preparation of Deodorant Stick Formulations Comprising the Invention's Composition

TABLE 12
Composition of Deodorant stick formulation

| Phase | Ingredients | Amount (% wt) |
|---|---|---|
| A | Stearic acid | 5.00 |
| | 1,2-Propylene glycol | 41.45 |
| | Sodium hydroxide (20% aqueous solution) | 4.20 |
| | Water | 30.00 |
| | Tetrasodium EDTA ⁽¹⁾ | 0.10 |
| | Ceteareth-25 ⁽²⁾ | 1.50 |
| | PPG-3 Myristyl ether ⁽³⁾ | 1.50 |
| B | 1,2-Propylene glycol | 15.00 |
| | Triclosan ⁽⁴⁾ | 0.25 |
| C | Perfume oil | 1 |

⁽¹⁾ Edeta ® B Power; origin: BASF
⁽²⁾ Cremophor ® A25; origin: BASF
⁽³⁾ Tegosoft ® APM; origin: Evonik
⁽⁴⁾ Irgasan ® DP 300; origin: BASF

Deodorant stick formulation is obtained by weighing all the components of Part A and heating to 70-75° C. Ceteareth-25 is added once the other Part A ingredients are mixed and heated. When the Ceteareth-25 is dissolved, stearic acid is added. Part B is prepared by dissolving Triclosan in 1,2-propylene glycol. Evaporated water is compensated. Then, slowly, under mixing, Part B is poured into Part A. A perfume oil being the invention's perfuming composition of Example 2 (Phase C) is added under gentle shaking. To stock, a plastic bag is put into the bucket to be sealed after cooling. Moulds were filled at about 70° C.

### Example 15

### Preparation of Deodorant Roll-on Formulations Comprising the Invention's Composition

TABLE 13
Composition of deodorant roll-on formulation

| Phase | Ingredients | Amount (% wt) |
|---|---|---|
| A | Water | 50.00 |
| | Hydroxyethylcellulose ⁽¹⁾ | 0.70 |
| B | Ethanol (95%) | 40.00 |
| | 1,2-Propylene glycol | 5.00 |
| | Triclosan ⁽²⁾ | 0.30 |
| C | PEG-40 hydrogenated castor oil ⁽³⁾ | 3.00 |
| D | Invention’s perfuming composition of Example 2 | 1 |

⁽¹⁾ Natrosol ® 250 H; origin: Ashland
⁽²⁾ Irgasan ® DP 300; origin: BASF
⁽³⁾ Cremophor ® RH 40; origin: BASF

Part A is prepared by sprinkling little-by-little the hydroxyethylcellulose into the water, whilst rapidly stirring with a turbine until the hydroxyethylcellulose is entirely swollen giving a limpid gel. Part B is slowly poured into Part A, whilst continuing stirring until the entire mixture is homogeneous. Then Parts C and D are added under gentle shaking.

### Example 16

### Preparation of Day Cream Base O/W Emulsions Comprising the Invention's Composition

TABLE 14
Composition of day cream base O/W emulsion formulation

| | | Amount |
|---|---|---|
| Phase | Ingredients | (% wt) |
| A | Steareth-2 (and) PEG-8 Distearate⁽¹⁾ | 5.0 |
| | Cetyl alcohol | 0.5 |
| | Ceteth-20 (AND) glyceryl stearate (and) | 4.0 |
| | PEG-6 stearate (and) Steareth-20 ⁽²⁾ | |
| | Squalan ⁽³⁾ | 1.0 |
| | Paraffin oil ⁽⁴⁾ | 2.0 |
| | Petrolatum ⁽⁵⁾ | 5.5 |
| B | Deionized water | 75.9 |
| | Propylene glycol | 5.0 |
| C | Phenoxyethanol (AND) Piroctone olamine ⁽⁶⁾ | 0.6 |
| D | Sodium carbomer ⁽⁷⁾ | 0.2 |
| E | Perfume oil | 0.3 |

⁽¹⁾Arlacel ® 985; origin: Croda
⁽²⁾ Tefose ® 2561; origin: Gattefossé
⁽³⁾ Biolip P 90; origin: Gattefossé
⁽⁴⁾ Mineral oil 30-40 CPS
⁽⁵⁾ Petroleum jelly
⁽⁶⁾ Nipaguard ® PO 5; origin: Clariant
⁽⁷⁾ PNC 400

Day cream base O/W emulsions is prepared by heating Phases A and B separately to 70-75° C. Phase A is added to Phase B, then vacuum is applied. The mixture is stirred and cooled to 55° C. for 15 min. After cooling to room temperature, phenoxyethanol (and) piroctone olamine (Part C) are added when a temperature of 45° C. is reached. The mixture is stirred for 5 min before sodium carbomer (Part D) and a perfume oil being the invention's perfuming composition of Example 2 (Part E) is added. The mixture is stirred for 3 min, then the stirring was stopped for 15 min. When the temperature of the mixture reaches 30° C., the stirring is resumed for another 15 min until the cream becomes homogeneous, glossy and without lumps. If necessary the pH is adjusted to 6.70-7.20 with Glydant, Phenonip or Nipaguard PO5 or to 6.30-7.00 with Nikkoguard.

### Example 17

### Performance of Invention's Compound as an Insect Repellant

1. Sample Preparation

Ethanolic solutions of a composition of matter as prepared in example 1 comprising (±)-3-(3-isopropyl-1-cyclopenten-1-yl)-2-methylpropanal, (±)-3-(4-isopropyl-1-cyclopenten-1-yl)-2-methylpropanal and 3-(3-isopropylcyclopentylidene)-2-methylpropanal (Stimulus A), a composition of matter as prepared in example 1 comprising (±)-2-methyl-3-[3-(2-methyl-2-propanyl)-1-cyclopenten-1-yl]propanal and (±)-2-methyl-3-[4-(2-methyl-2-propanyl)-1-cyclopenten-1-yl]propanal (Stimulus B), a composition of matter as prepared in example 1 comprising (±)-3-(3-ethyl-1-cyclopenten-1-yl)-2-methylpropanal and (±)-3-(4-ethyl-1-cyclopenten-1-yl)-2-methylpropanal (Stimulus C), a composition of matter as prepared in example 1 comprising (±)-3-(4-butyl-1-cyclopenten-1-yl)-2-methylpropanal and (±)-3-(3-butyl-1-cyclopenten-1-yl)-2-methylpropanal (Stimulus D), a composition of matter as prepared in example 1 comprising (±)-2-methyl-3-(3-propyl-1-cyclopenten-1-yl)propanal and (±)-2-methyl-3-(4-propyl-1-cyclopenten-1-yl)propanal (Stimulus E) and a composition of matter as prepared in example 1 comprising (±)-3-(3-sec-butyl-1-cyclopenten-1-yl)-2-methylpropanal and (±)-3-(4-sec-butyl-1-cyclopenten-1-yl)-2-methylpropanal (Stimulus F) at nine concentrations ranging between 0.00032% and 1% (w/w) were used for the in vitro test.

2. Model Organism

Controlling efficacy was tested against the yellow fever mosquito, Aedes aegypti Rockefeller strain.

Aedes aegypti is a model organism for controlling tests and one of the recommended model organisms by the World Health Organization (WHO) as it is a very aggressive, anthropophilic mosquito species that shows generally low sensitivity to arthropod controlling compounds.

Observations of controlling efficacy were made on host-seeking females of uniform age, 5 to 12 days post-eclosion from pupae. Tested hungry females had access to 10% sugar solution but were not blood-fed.

3. In Vitro Warm Body Assay for Flying Arthropods Such as Mosquitoes:

The controlling effect according to the present invention was assessed using an adapted Warm Body assay (WBA) as defined in Kröber T, Kessler S, Frei J, Bourquin M, Guerin P M. An in vitro assay for testing mosquito controlling compounds employing a warm body and carbon dioxide as a behavioral activator. J Am Mosq Control Assoc. 2010; 26:381-386.

The published protocol has been adapted in not manually counting the landing mosquitoes but automatically using an automatic counting software, the switch from Anopheles gambiae to Aedes aegypti led to a decrease of mosquitoes' number placed in the tested cage due to the size difference (i.e. 30 mosquitoes instead of 50) and to an increase of lighting as Aedes aegypti is a diurnal mosquito (i.e. 150 lux instead of 4 lux).

In this in-vitro assay the number of Aedes aegypti landing on a warm body treated with the tested stimuli was measured in order to assess the repellency effect.

4. Evaluation of the Arthropod Controlling Effect of the Present Invention

The in-vitro mean numbers of mosquitoes Aedes aegypti landing on the Warm Body loaded with different dilutions of a composition of matter as prepared in example 1 comprising (±)-3-(3-isopropyl-1-cyclopenten-1-yl)-2-methylpropanal and (±)-3-(4-isopropyl-1-cyclopenten-1-yl)-2-methylpropanal (Stimulus A), a composition of matter as prepared in example 1 comprising (±)-2-methyl-3-[3-(2-methyl-2-propanyl)-1-cyclopenten-1-yl]propanal and (±)-2-methyl-3-[4-(2-methyl-2-propanyl)-1-cyclopenten-1-yl]propanal (Stimulus B), a composition of matter as prepared in example 1 comprising (±)-3-(3-ethyl-1-cyclopenten-1-yl)-2-methylpropanal and (±)-3-(4-ethyl-1-cyclopenten-1-yl)-2-methylpropanal (Stimulus C), a composition of matter as prepared in example 1 comprising (±)-3-(4-butyl-1-cyclopenten-1-yl)-2-methylpropanal and (±)-3-(3-butyl-1-cyclopenten-1-yl)-2-methylpropanal (Stimulus D), a composition of matter as prepared in example 1 comprising (±)-2-methyl-3-(3-propyl-1-cyclopenten-1-yl)propanal and (±)-2-methyl-3-(4-propyl-1-cyclopenten-1-yl)propanal (Stimulus E) and a composition of matter as prepared in example 1 comprising (±)-3-(3-sec-butyl-1-cyclopenten-1-yl) methylpropanal and (±)-3-(4-sec-butyl-1-cyclopenten-1-yl)-2-methylpropanal (Stimulus F) is displayed in the following Table 15.

TABLE 15
number of mosquitoes Aedes aegypti landing within 2 min test
period after application of different dilutions of Stimulus
A to F in the in-vitro WBA. Number of landing with ethanol was
66 ± 7, 65 ± 8, 69 ± 5, 56 ± 3, 62 ± 3 & 82 ± 15 for stimuli
A, B, C, D, E & F respectively.

| | number of mosquito landing | | | | | |
|---|---|---|---|---|---|---|
| % | Stimulus | Stimulus | Stimulus | Stimulus | Stimulus | Stimulus |
| concentration | A | B | C | D | E | F |
| 0.00032 | 35 | 56 | 71 | 97 | 62 | 72 |
| 0.0016 | 63 | 55 | 44 | 56 | 10 | 38 |
| 0.00357 | 102 | 60 | 83 | 84 | 100 | 71 |
| 0.008 | 26 | 69 | 78 | 89 | 0 | 87 |
| 0.0178 | 73 | 0 | 33 | 82 | 79 | 63 |
| 0.04 | 38 | 17 | 15 | 13 | 27 | 10 |
| 0.0896 | 66 | 10 | 30 | 11 | 24 | 35 |
| 0.2 | 5 | 44 | 0 | 0 | 0 | 24 |
| 1 | 7 | 6 | 13 | 1 | 17 | 9 |

The number of mosquitoes Aedes aegypti landing on the warm body decreases with the increase tested amounts of stimulus demonstrating the biological effect of the composition of matter as prepared in example 1 comprising (±)-3-(3-isopropyl-1-cyclopenten-1-yl)-2-methylpropanal and (±)-3-(4-isopropyl-1-cyclopenten-1-yl)-2-methylpropanal (Table 15). The number of mosquitoes Aedes aegypti landing on the warm body decreases with the increase tested amounts of stimulus demonstrating the biological repellent effect of all the six tested compositions of matter (Table 15).

At concentration of stimuli equal to 1%, the repulsion was consistently higher with a reduction of 89% of landings on the warm body loaded with the tested compound (Table 1). At concentration of stimuli equal to 1%, the repulsion was consistently higher with a reduction of 89%, 91%, 82%, 98%, 73% & 89% of landings on the warm body loaded with the tested stimuli A, B, C, D, E & F respectively (Table 15).

### Example 18

### Performance of Invention's Compound as an Antimicrobial—Minimum Inhibitory Concentration (MIC) Test

1. MIC Results

The below MIC results show that the composition of matter as prepared in example 1 comprising (±)-3-(3-isopropyl-1-cyclopenten-1-yl)-2-methylpropanal, (±)-3-(4-isopropyl-1-cyclopenten-1-yl)-2-methylpropanal and 3-(3-isopropylcyclopentylidene) methylpropanal is highly active for Staphylococcus aureus and Cutibacterium acnes as shown in below Table 16.

TABLE 16
Minimal inhibitory concentration of the composition of
matter as prepared in example 1 comprising
(+)-3-(3-isopropyl-1-cyclopenten-1-yl)-2-methylpropanal, (±)-3-
(4-isopropyl-1-cyclopenten-1-yl)-2-methylpropanal
and 3-(3-isopropylcyclopentylidene)-
2-methylpropanal on bacterial strains.

| Staphylococcus aureus | Cutibacterium acnes |
|---|---|
| ATCC 6538 | DSM 1897 |
| 333 ppm | 148 ppm |

2. Method

### Preparation of Bacterial Suspensions

Bacterial suspensions of Staphylococcus aureus ATCC 6538 were prepared as follows. Stock cultures stored at −80° C. were subcultured onto agar plate media, and incubated at 37° C. for 24 h to obtain single colonies. Single colonies of the primary cultures were inoculated into broth media and incubated at 37° C., 160 rpm overnight. Aliquots of overnight cultures were inoculated into 50 ml of fresh broth media, and incubated at 37° C., 160 rpm. When the OD_{600 nm} reached the target value for each strain (see Table 17), cells were harvested by centrifugation at 5000 rpm for 10 min, and then resuspended in the same fresh broth media at the same volume before the centrifugation. Aliquots (1.1 ml) of each cell suspension were diluted in 200 ml of the same broth media as the bacterial solutions for the MIC test.

TABLE 17
Media, aliquots of overnight culture, and target OD of broth
cultures for the preparation of bacterial suspensions of S. aureus

| | | | Aliquots of | |
|---|---|---|---|---|
| | Agar plate | Broth | Overnight | Target |
| Strains | media | Media | Culture | OD |
| Staphylococcus aureus | TSA | MH | 0.5 ml | 0.4 |
| ATCC 6538 | | | | |

Note:
^{a}TSA, Tryptic Soy Agar (BD Cat No. 236950),
^{b}MH, Mueller Hinton Broth (BD Cat No. 211443)

Bacterial solutions of anaerobic Cutibacterium acnes DSM 1897 were prepared for MIC test as follows. Stock culture stored at −80° C. were subcultured onto Columbia blood agar (CBA, Biomerieux) plates, and incubated at 37° C. anaerobically for 4 d to obtain single colonies. One loop (1 uL) of the 4 d culture to 9.5 mL Wilkins-Chalgren broth (WC, OXOID) and incubated at 37° C., anerobically for 24 h. Aliquots (2 mL) of the 24 h cultures were inoculated into 100 ml of fresh WC broth media, and incubated at 37° C., anaerobically for 18 h. Aliquots (1.4 ml) of the overnight culture were diluted in 100 ml of WC broth media as the bacterial solutions for the MIC test.

### Preparation of Sample Solutions

(2E)-2-hexenal was used as the reference material for MIC test of aerobic strains, whereas carvacrol was used for anaerobic strains.

Sample solutions of test materials and reference material were prepared in ethanol for MIC test for S. aureus or in DMSO for C. acnes. In brief, stock solutions of 1% and 20% were prepared in each solvent, and then seven serial dilutions of each stock solution were prepared in each solvent to obtain a total of 16 solutions of the test material or the reference material. Aliquot (10 μl) of each solution was used for MIC test. The tested final concentrations of each material were 29, 44, 66, 99, 148, 222, 333, 500, 590, 900, 1300, 2000, 3000, 4500, 6700, 10000 ppm.

### MIC Test Procedures

MIC test were performed in 96 well plates. Table 18 shows the schematic positions of sample solutions in 96 well plates. Column 1 contained bacterial solution only (the positive growth control), and column 12 contained growth media only (the negative growth control). Aliquots (10 μl) of sample solutions were mixed with 190 μl of bacterial solutions in growth media, at concentrations of 10⁵ to 10⁶ cfu/ml, in wells of the 96 well plates. Three replicates for each solution.

The 96 wells plates were incubated at 37° C. for 1 day for S. aureus and 2 days for C. acnes. After incubation, wells of 96 well plates were examined Turbid wells were regarded as an indication of microbial growth.

Minimal inhibitory concentration (MIC) was determined as the lowest concentration where no growth was observed. Average MIC value of the three replicates was calculated against each strain.

TABLE 18
Schematic positions of samples in 96 well plates

| A | Positive | | | 590 ppm | 29 ppm | | | 590 ppm | 29 ppm | | | Negative |
|---|---|---|---|---|---|---|---|---|---|---|---|---|
| B | growth | | | 900 ppm | 44 ppm | | | 900 ppm | 44 ppm | | | growth |
| C | control | | | 1300 ppm | 66 ppm | | | 1300 ppm | 66 ppm | | | control |
| D | | | | 2000 ppm | 99 ppm | | | 2000 ppm | 99 ppm | | | |
| E | | | | 3000 ppm | 148 ppm | | | 3000 ppm | 148 ppm | | | |
| F | | | | 4500 ppm | 222 ppm | | | 4500 ppm | 222 ppm | | | |
| G | | | | 6700 ppm | 333 ppm | | | 6700 ppm | 333 ppm | | | |
| H | | | | 10000 ppm | 500 ppm | | | 10000 ppm | 500 ppm | | | |
| | 1 | 2 | 3 | 4 | 5 | 6 | 7 | 8 | 9 | 10 | 11 | 12 |

| | |
|---|---|
| Test | Reference |
| material | material |

1. A compound of formula (I): 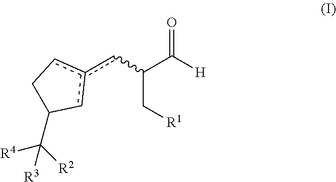
in the form of any one of its stereoisomers or as a mixture thereof, wherein one dotted line represents a carbon-carbon double bond and the other dotted lines represent a carbon-carbon single bond; R¹ represents a hydrogen atom or a C₁₋₂ alkyl group; R² represents a C₁₋₄ alkyl group or a C₂₋₄ alkenyl group; R³ represents a hydrogen atom or a C₁₋₃ alkyl group or a C₂₋₃ alkenyl group; R⁴ represents a hydrogen atom or R⁴ represents a hydrogen atom or a methyl group when R² and R³ represent a methyl group; or R³ and R⁴, when taken together, represent a methylidene group; or R² and R³, when taken together, represent a C₄₋₉ alkanediyl or a C₄₋₉ alkenediyl group; or R², R³ and R⁴, when taken together, represent a phenyl group optionally substituted by a C₁₋₃ alkyl group.

2. The compound according to claim 1, wherein the compound is of formula (II): 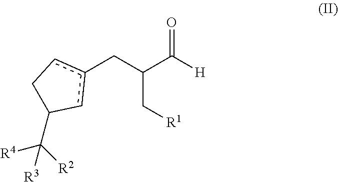
in the form of any one of its stereoisomers or as a mixture thereof, wherein one dotted line represents a carbon-carbon double bond and the other dotted line represents a carbon-carbon single bond; R¹, R², R³ and R⁴ have the same meaning as defined in claim 1.

3. The compound according to claim 1, wherein R¹ and R⁴ are a hydrogen atom.

4. The compound according to claim 1, wherein R³ is a hydrogen atom or a methyl group.

5. The compound according to claim 1, wherein R² is a methyl, ethyl or propyl group.

6. The compound according to claim 1, wherein the compound of formula (I) is selected from the group consisting of 3-(3/4-ethylcyclopent-1-en-1-yl)-2-methylpropanal, 3-(3/4-propylcyclopent-1-en-1-yl)-2-methylpropanal, 3-(3/4-butylcyclopent-1-en-1-yl)-2-methylpropanal, 3-(3/4-isopropylcyclopent-1-en-1-yl)-2-methylpropanal, 3-(3/4-(sec-butyl)cyclopent-1-en-1-yl)-2-methylpropanal and 3-(3/4-(tert-butyl)cyclopent-1-en-1-yl)-2-methylpropanal.

7. (canceled)

8. A method to confer, enhance, improve or modify the odor properties of a perfuming composition or of a perfumed article, which method comprises adding to said composition or article an effective amount of at least one compound of formula (I) as defined in claim 1.

9. A properfume compound suitable to release the compound of formula (I): 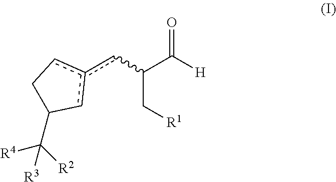
in the form of any one of its stereoisomers or as a mixture thereof, wherein one dotted line represents a carbon-carbon double bond and the other dotted lines represent a carbon-carbon single bond; le represents a hydrogen atom or a C₁₋₂ alkyl group; R² represents a C₁₋₄ alkyl group or a C₂₋₄ alkenyl group; R³ represents a hydrogen atom or a C₁₋₃ alkyl group or a C₂₋₃ alkenyl group; R⁴ represents a hydrogen atom or R⁴ represents a hydrogen atom or a methyl group when R² and R³ represent a methyl group; or R³ and R⁴, when taken together, represent a methylidene group; or R² and R³, when taken together, represent a C₄₋₉ alkanediyl or a C₄₋₉ alkenediyl group; or R², R³ and R⁴, when taken together, represent a phenyl group optionally substituted by a C₁₋₃ alkyl group.

10. A perfuming composition comprising
i) at least one compound of formula (I), as defined in claim 1;
ii) at least one ingredient selected from the group consisting of a perfumery carrier and a perfumery base; and
iii) optionally at least one perfumery adjuvant.

11. A perfumed consumer product comprising at least one compound of formula (I), as defined in claim 1.

12. The perfumed consumer product according to claim 11, characterized in that the perfumery consumer product is a perfume, a fabric care product, a body-care product, a cosmetic preparation, a skin-care product, an air care product or a home care product.

13. The perfumed consumer product according to claim 12, characterized in that the perfumery consumer product is a fine perfume, a splash or eau de parfum, a cologne, a shave or after-shave lotion, a liquid or solid detergent, a fabric softener, a fabric refresher, an ironing water, a paper, a bleach, a carpet cleaner, a curtain-care product, a shampoo, a coloring preparation, a color care product, a hair shaping product, a dental care product, a disinfectant, an intimate care product, a hair spray, a vanishing cream, a deodorant or antiperspirant, a hair remover, a tanning or sun product, a nail product, a skin cleansing, a makeup, a perfumed soap, a shower or bath mousse, oil or gel, or a foot/hand care product, a hygiene product, an air freshener, a “ready to use” powdered air freshener, a mold remover, a furnisher care, a wipe, a dish detergent or hard-surface detergent, a leather care product, a car care product, a pest control article, an insect repellent product.

14. A method to confer, enhance, improve or modify the arthropod control properties of an arthropod control composition or of an arthropod control article, which method comprises adding to said composition or article an effective amount of at least a compound of formula (I) as defined in claim 1.

15. A non-therapeutic method of reducing, arresting or inhibiting the growth rate of bacterial cells or inactivating or killing bacterial cells, the method comprising treating a substrate comprising microbes with an effective amount of at least a compound of formula (I) as defined in claim 1.

16. The compound according to claim 2, wherein:
R¹ and R⁴ are a hydrogen atom;
R³ is a hydrogen atom or a methyl group; and
R² is a methyl, ethyl or propyl group.

17. A perfuming composition comprising
i) at least one properfume compound as defined in claim 9;
ii) at least one ingredient selected from the group consisting of a perfumery carrier and a perfumery base; and
iv) optionally at least one perfumery adjuvant.

18. A perfumed consumer product comprising at least one properfume compound as defined in claim 9.

19. The perfumed consumer product according to claim 18, characterized in that the perfumery consumer product is a perfume, a fabric care product, a body-care product, a cosmetic preparation, a skin-care product, an air care product or a home care product.

20. The perfumed consumer product according to claim 19, characterized in that the perfumery consumer product is a fine perfume, a splash or eau de parfum, a cologne, a shave or after-shave lotion, a liquid or solid detergent, a fabric softener, a fabric refresher, an ironing water, a paper, a bleach, a carpet cleaner, a curtain-care product, a shampoo, a coloring preparation, a color care product, a hair shaping product, a dental care product, a disinfectant, an intimate care product, a hair spray, a vanishing cream, a deodorant or antiperspirant, a hair remover, a tanning or sun product, a nail product, a skin cleansing, a makeup, a perfumed soap, a shower or bath mousse, oil or gel, or a foot/hand care product, a hygiene product, an air freshener, a “ready to use” powdered air freshener, a mold remover, a furnisher care, a wipe, a dish detergent or hard-surface detergent, a leather care product, a car care product, a pest control article, an insect repellent product.
